(19) United States
(12) Patent Application Publication
(10) Pub. No.: US 2026/0199277 A1
(43) Pub. Date: Jul. 16, 2026

(54) Therapeutic Compositions for the Treatment of Bacterial Vaginosis and Methods of Use Thereof

(71) Applicants: The General Hospital Corporation, Boston, MA (US); Massachusetts Institute of Technology, Cambridge, MA (US); The Broad Institute, Inc., Cambridge, MA (US)

(72) Inventors: Douglas S. Kwon, Somerville, MA (US); Paul Blainey, Cambridge, MA (US); Seth M. Bloom, Brookline, MA (US); Meilin Zhu, Cambridge, MA (US)

(21) Appl. No.: 19/552,306

(22) Filed: Feb. 27, 2026

Related U.S. Application Data
(63) Continuation-in-part of application No. PCT/US2024/044631, filed on Aug. 30, 2024.
(60) Provisional application No. 63/536,186, filed on Sep. 1, 2023.
(60) Provisional application No. 63/570,876, filed on Mar. 28, 2024.
(60) Provisional application No. 63/764,519, filed on Feb. 27, 2025.

(51) Int. Cl. A61K 31/201; A61K 31/4164; A61K 31/7056; A61K 45/06; A61P 31/04

(52) U.S. Cl. CPC A61K 31/201; A61K 31/4164; A61K 31/7056; A61K 45/06; A61P 31/04

(57) ABSTRACT

Described herein are compositions for treatment of vaginal dysbiosis (e.g., bacterial vaginosis), symptoms of vaginal dysbiosis (e.g., bacterial vaginosis), or conditions/disorders associated with vaginal dysbiosis (e.g., bacterial vaginosis). Also disclosed herein are methods for treating vaginal dysbiosis (e.g., bacterial vaginosis) in an individual in need thereof, methods for reducing the risk of vaginal dysbiosis (e.g., bacterial vaginosis) in an individual in need thereof, methods for preventing recurrence of vaginal dysbiosis (e.g., bacterial vaginosis) in an individual treated for a prior episode of vaginal dysbiosis (e.g., bacterial vaginosis). Also described are methods for restoring Lactobacillus dominated microbiota, including L. crispatus-dominated microbiota or microbiota dominated by other non-iners Lactobacillus species, in an individual in need thereof.

### CROSS-REFERENCE TO RELATED APPLICATIONS

This application is a continuation-in-part of and claims benefit under 35 U.S.C. § 120 from PCT Application No. PCT/US2024/044631, filed on Aug. 30, 2024, which claims priority to U.S. Provisional Application No. 63/536,186, filed on Sep. 1, 2023, and U.S. Provisional Application No. 63/570,876, filed on Mar. 28, 2024. This application also claims priority to U.S. Provisional Application No. 63/764,519, filed on Feb. 27, 2025. The entire contents of each of these earlier applications are incorporated herein by reference.

### FEDERALLY SPONSORED RESEARCH OR DEVELOPMENT

This invention was made with Government support under Grant Nos. AI171166, AI166116, and AI42780 awarded by the National Institutes of Health; and Grant No. 1745302 awarded by the National Science Foundation. The Government has certain rights in the invention.

### TECHNICAL FIELD

Described herein are compositions for treatment of bacterial vaginosis, symptoms of bacterial vaginosis, or conditions/disorders associated with bacterial vaginosis.

### BACKGROUND

The composition of the female genital tract (FGT) microbiome strongly influences women's reproductive health outcomes, including development of bacterial vaginosis (BV), risk of preterm birth, genital inflammation/vaginitis, cervical cancer, and susceptibility to sexually transmitted diseases, including HIV. Health-associated FGT microbiome states typically exhibit dominance by a single species of Lactobacillus (e.g. Lactobacillus crispatus, Lactobacillus gasseri, Lactobacillus jensenii, or Lactobacillus mulieris), while disease-associated states comprise more diverse obligate anaerobic microbial species with low Lactobacillus abundance. Interestingly, microbiota states dominated by Lactobacillus iners (unlike other non-iners FGT lactobacilli) have been linked to higher prevalence of cervical dysplasia and are strongly associated with transition to more disease-associated microbiota states, including clinical bacterial vaginosis (BV). BV is a highly prevalent vaginal syndrome characterized by abundance of obligate anaerobic bacteria. BV affects 20-30% of women around the world and the costs associated with BV and its sequelae are an estimated 13 billion US dollars annually. Treatment with antibiotics—the standard of care for BV—typically shifts communities towards L. iners-dominance rather than dominance by more optimally health-associated species such as L. crispatus. L. iners-dominance after BV treatment likely contributes to high rates of recurrence; over 50% of women treated for BV experience recurrence by 12 months post-treatment. Additionally, key BV associated anaerobic bacteria such as Gardnerella or Fannyhessea (formerly Atopobium) can be metronidazole or clindamycin resistant, further contributing to recurrence in some patients. Recent strategies using L. crispatus as a living biotherapeutic have shown only moderate success, likely owing to the difficulty of manipulating female genital tract microbiome composition and establishing successful colonization by the living biotherapeutic strains used. Thus, novel approaches to shift BV-like communities towards non-iners Lactobacillus-dominance, through selective inhibition of L. iners and BV-associated species and growth facilitation of health-associated ones, may provide new and effective treatment options for BV and reduce risk for multiple additional important adverse health outcomes.

An effective clinical intervention to durably shift the vaginal microbiota to Lactobacillus dominance—and ideally, L. crispatus dominance in a durable fashion - would allow improved clinical treatment of women with recurrent BV, which is associated with adverse health outcomes and has a significant negative impact on quality of life. In addition, if such an intervention promoted durable L. crispatus dominance, it could potentially be used to decrease rates of adverse health outcomes, including of preterm birth, HIV acquisition, cervical dysplasia, post-operative gynecologic infections and increase the success of in vitro fertilization and spontaneous conception, all of which would provide a significant public health benefit.

### SUMMARY

It has now been determined that unsaturated long chain fatty acids (U-LCFAs) or their metabolic derivatives (referred to also as “hydroxylated derivative” or “hydroxylated metabolic derivative”) selectively inhibit growth of L. iners, Gardnerella spp., Fannyhessea vaginae, and Sneathia amnii and that at least the U-LCFA oleic acid selectively enhances the growth of the non-iners lactobacilli L. crispatus, L. gasseri, L. jensenii, and L. mulieris.

In some embodiments, provided herein are methods of treating vaginal dysbiosis in a subject, said method comprising administering to the subject a composition comprising an active ingredient comprising or consisting of one or more unsaturated long chain fatty acids or metabolic derivative thereof (e.g., a hydroxylated derivative thereof) in an amount and duration effective to increase vaginal colonization of non-Lactobacillus-iners species, thereby treating the vaginal dysbiosis in the subject.

In some embodiments, provided herein are methods of reducing the risk of a subject developing vaginal dysbiosis, said method comprising administering to the subject a composition comprising an active ingredient comprising or consisting of one or more unsaturated long chain fatty acids or metabolic derivative thereof (e.g., a hydroxylated derivative thereof) in an amount and duration effective to increase vaginal colonization of non-Lactobacillus-iners species, thereby reducing the risk of the subject developing vaginal dysbiosis.

In some instances of any of the methods described above, the vaginal dysbiosis is bacterial vaginosis.

Also provided herein are methods of treating a subject having Lactobacillus iners-dominant vaginal colonization, said method comprising administering to the subject a composition comprising an active ingredient comprising or consisting of one or more unsaturated long chain fatty acids or metabolic derivative thereof (e.g., a hydroxylated derivative thereof) in an amount and duration effective to increase vaginal colonization of Lactobacillus-crispatus and decrease vaginal colonization of Lactobacillus-iners, thereby treating the subject.

Also described herein are methods of reducing the risk of a subject developing Lactobacillus iners-dominant vaginal colonization, said method comprising administering to the subject a composition comprising an active ingredient comprising or consisting of one or more unsaturated long chain fatty acids or metabolic derivative thereof (e.g., a hydroxylated derivative thereof) in an amount and duration effective to increase vaginal colonization of Lactobacillus-crispatus and decrease vaginal colonization of Lactobacillus-iners, thereby reducing the risk of the subject developing Lactobacillus iners-dominant vaginal colonization.

In some instances of any of the methods described above, the one or more unsaturated long chain fatty acid or metabolic derivative thereof is selected from oleic acid, myristoleic acid, palmitoleic acid, linoleic acid, linolenic acid, gamma-linolenic acid, cis-11-eicosenoic acid, erucic acid, elaidic acid, eicosapentaenoic acid, 11,14,17-eicosatrienoic acid, mead acid, nervonic acid, pinolenic acid, stearidonic acid, sapienic acid, cis-vaccenic acid, cis-4,7,10,13,16,19-docosahexaenoic acid, arachidonic acid, 10-hydroxymyristic acid, 10-hydroxypalmitic acid, 10-hydroxystearic acid, 10-hydroxy-12-octadecenoic acid, and 13-hydroxy-9-octadecenoic acid or combinations thereof.

In some instances, the one or more unsaturated long chain fatty acid is oleic acid. In some instances, the one or more or metabolic derivative thereof is selected from 10-hydroxymyristic acid, 10-hydroxypalmitic acid, 10-hydroxystearic acid, 10-hydroxy-12-octadecenoic acid, and 13-hydroxy-9-octadecenoic acid or combinations thereof. In some instances, the one or more unsaturated long chain fatty acid or metabolic derivative thereof comprises a combination of oleic acid and 10-hydroxystearic acid.

In some instances, the one or more unsaturated long chain fatty acid or metabolic derivative thereof can be a synergistic combination for inhibiting L. iners growth comprising, or consisting of elaidic acid and 10-hydroxystearic acid, elaidic acid and palmitoleic acid, elaidic acid and linolenic acid, elaidic acid and oleic acid, cis-vaccenic acid and palmitoleic, cis-vaccenic acid and oleic acid, and 10-hydroxystearic acid and 10-hydroxy-12-octadecenoic acid.

In some instances, the one or more unsaturated long chain fatty acid or metabolic derivative thereof can be a synergistic combination for increasing L. crispatus growth comprising, or consisting of, cis-vaccenic acid and 10-hydroxystearic acid, elaidic acid and linolenic acid, 10-hydroxystearic acid and myristoleic acid, elaidic acid and palmitoleic acid, and elaidic acid and linolenic acid.

In some instances, the one or more unsaturated long chain fatty acid or metabolic derivative thereof can be a combination which inhibits diverse bacterial species contributing to bacterial vaginosis comprising, or consisting of, elaidic acid and 10-hydroxystearic acid, cis-vaccenic acid and elaidic acid, and cis-vaccenic acid and 10-hydroxystearic acid.

In some instances of any of the methods described above, the composition comprising an active ingredient comprising or consisting of the one or more unsaturated long chain fatty acids or metabolic derivative thereof is administered following, or prior to, administration of an antimicrobial agent to the subject. In some instances, the subject was previously administered the antimicrobial agent and is concurrently administered one or more additional doses of the antimicrobial agent with the unsaturated long chain fatty acid or metabolic derivative thereof. In some instances, the antimicrobial agent is metronidazole or clindamycin. In some instances, the subject was previously administered the antimicrobial agent and is concurrently administered a different antimicrobial agent with the unsaturated long chain fatty acid or metabolic derivative thereof.

In some instances of any of the methods described above, the method further comprises administration of one or more live biotherapeutics (LBPs).

In some instances of any of the methods described above, the active ingredient consists of the one or more unsaturated long chain fatty acids or metabolic derivative thereof.

In some instances of any of the methods described above, the composition is administered to the subject orally, rectally, or vaginally.

Also described herein is a composition comprising an active ingredient consisting of one or more unsaturated long chain fatty acids or metabolic derivative thereof.

In some instances, the one or more unsaturated long chain fatty acid or metabolic derivative thereof is selected from oleic acid, myristoleic acid, palmitoleic acid, linoleic acid, linolenic acid, gamma-linolenic acid, cis-11-eicosenoic acid, erucic acid, elaidic acid, eicosapentaenoic acid, 11,14,17-eicosatrienoic acid, mead acid, nervonic acid, pinolenic acid, stearidonic acid, sapienic acid, cis-4,7,10,13,16,19-docosahexaenoic acid, arachidonic acid, 10-hydroxymyristic acid, 10-hydroxypalmitic acid, 10-hydroxystearic acid, 10-hydroxy-12-octadecenoic acid, and 13-hydroxy-9-octadecenoic acid or combinations thereof.

In some instances, the one or more unsaturated long chain fatty acid is oleic acid.

In some instances, the one or more unsaturated long chain fatty acid or metabolic derivative thereof is selected from 10-hydroxymyristic acid, 10-hydroxypalmitic acid, 10-hydroxystearic acid, 10-hydroxy-12-octadecenoic acid, and 13-hydroxy-9-octadecenoic acid or combinations thereof.

In some instances, the one or more unsaturated long chain fatty acid or metabolic derivative thereof comprises a combination of oleic acid and 10-hydroxystearic acid.

In some instances, the one or more unsaturated long chain fatty acid or metabolic derivative thereof can be a synergistic combination for inhibiting L. iners growth comprising, or consisting of elaidic acid and 10-hydroxystearic acid, elaidic acid and palmitoleic acid, elaidic acid and linolenic acid, elaidic acid and oleic acid, cis-vaccenic acid and palmitoleic, cis-vaccenic acid and oleic acid, and 10-hydroxystearic acid and 10-hydroxy-12-octadecenoic acid.

In some instances, the one or more unsaturated long chain fatty acid or metabolic derivative thereof can be a synergistic combination for increasing L. crispatus growth comprising, or consisting of, cis-vaccenic acid and 10-hydroxystearic acid, elaidic acid and linolenic acid, 10-hydroxystearic acid and myristoleic acid, elaidic acid and palmitoleic acid, and elaidic acid and linolenic acid.

In some instances, the one or more unsaturated long chain fatty acid or metabolic derivative thereof can be a combination which inhibits diverse bacterial species contributing to bacterial vaginosis comprising, or consisting of, elaidic acid and 10-hydroxystearic acid, cis-vaccenic acid and elaidic acid, and cis-vaccenic acid and 10-hydroxystearic acid.

In some instances, the composition further comprises one or more

pharmaceutically acceptable excipients.

In some instances, the composition is formulated for oral, rectal, or vaginal administration.

In one aspect, the invention provides a method of treating a subject having Lactobacillus iners-dominant vaginal colonization, said method comprising administering to the subject one or more unsaturated long chain fatty acids in an amount and duration effective to increase vaginal colonization of Lactobacillus-crispatus and decrease vaginal colonization of Lactobacillus-iners following, or prior to, administration of an antimicrobial agent to the subject, thereby treating the subject.

In one embodiment, the unsaturated long chain fatty acid or metabolic derivative thereof is selected from myristoleic acid, palmitoleic acid, oleic acid, linoleic acid, and their hydroxylated metabolic products (respectively, 10-hydroxymyristic acid, 10-hydroxypalmitic acid, 10-hydroxystearic acid, 10-hydroxy-12-octadecenoic acid, and 13-hydroxy-9-octadecenoic acid) or combinations thereof.

In another embodiment, the antimicrobial agent is metronidazole or clindamycin.

In yet another embodiment, the subject was previously administered the antimicrobial agent and is now concurrently administered an antimicrobial agent with the unsaturated long chain fatty acid.

In yet another embodiment, the subject having Lactobacillus iners-dominant vaginal colonization has bacterial vaginosis.

In yet another embodiment, the subject has been identified as having Lactobacillus iners-dominant vaginal colonization.

In another aspect, the invention provides a method of treating bacterial vaginosis in a subject, said method comprising administering to the subject one or more unsaturated long chain fatty acids in an amount and duration effective to increase vaginal colonization of Lactobacillus-crispatus and decrease vaginal colonization of Lactobacillus-iners following, or prior to, administration of an antimicrobial agent to the subject, thereby treating the bacterial vaginosis in the subject.

In one embodiment, the unsaturated long chain fatty acid or metabolic derivative thereof is selected from myristoleic acid, palmitoleic acid, oleic acid, linoleic acid, and their hydroxylated metabolic products (10-hydroxymyristic acid, 10-hydroxypalmitic acid, 10-hydroxystearic acid, 10-hydroxy-12-octadecenoic acid, and 13-hydroxy-9-octadecenoic acid) or combinations thereof.

In another embodiment, the antimicrobial agent is metronidazole or clindamycin.

In yet another embodiment, the subject was previously administered the antimicrobial agent and is now concurrently administered an antimicrobial agent with the unsaturated long chain fatty acid.

In yet another embodiment, the subject having Lactobacillus iners-dominant vaginal colonization has bacterial vaginosis.

In yet another aspect, the invention provides a method of reducing the risk of a subject developing Lactobacillus iners-dominant vaginal colonization, said method comprising administering to the subject one or more unsaturated long chain fatty acids or metabolic derivative thereof in an amount and duration effective to increase vaginal colonization of Lactobacillus-crispatus and decrease vaginal colonization of Lactobacillus-iners, thereby reducing the risk of the subject developing Lactobacillus iners-dominant vaginal colonization.

In one embodiment, the unsaturated long chain fatty acid or metabolic derivative thereof is selected from myristoleic acid, palmitoleic acid, oleic acid, linoleic acid,, and their hydroxylated metabolic products (10-hydroxymyristic acid, 10-hydroxypalmitic acid, 10-hydroxystearic acid, 10-hydroxy-12-octadecenoic acid, and 13-hydroxy-9-octadecenoic acid) or combinations thereof.

In another embodiment, the antimicrobial agent is metronidazole or clindamycin.

In yet another embodiment, the subject was previously administered the antimicrobial agent and is now concurrently administered an antimicrobial agent with the unsaturated long chain fatty acid.

In yet another embodiment, the subject is at risk of developing a bacterial vaginosis infection.

Other features and advantages of the invention will be apparent from the Detailed Description, and from the claims. Thus, other aspects of the invention are described in the following disclosure and are within the scope of the invention.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

The following Detailed Description, given by way of example, but not intended to limit the invention to specific embodiments described, may be understood in conjunction with the accompanying figures, incorporated herein by reference.

FIG. 1 depicts structures of example relevant unsaturated long chain fatty acids (U-LCFAs) and their hydroxylated derivatives that selectively inhibit disease-associated and facilitate health-associated FGT bacterial species.

FIG. 2 depicts U-LCFAs including myristoleic acid (MOA), palmitoleic acid (POA), oleic acid (OA), and linoleic acid (LOA) inhibiting three representative strains of L. iners at significantly lower concentrations compared to representative strains of L. crispatus, L. gasseri, L. jensenii, and L. mulieris.

FIG. 3 depicts Oleic acid selectively inhibiting 14 distinct strains of L. iners compared to 32 distinct strains of L. crispatus, L. gasseri, L. jensenii, and L. mulieris in Lactobacillus-optimized modified MRS medium (left panel), of oleic acid selectively promoting the growth of 32 strains of these non-iners Lactobacillus species at equivalent concentrations while also inhibiting L. iners in a serum-rich media (S-broth) that is less well-adapted to support growth of lactobacilli (middle panel), and of oleic acid exerting bactericidal activity against L. iners but not the non-iners Lactobacillus species in modified MRS medium at equivalent concentrations (right).

FIG. 4 depicts oleic acid selectively inhibiting 14 distinct strains of L. iners while promoting growth of 13 distinct strains of L. crispatus in an additional serum-based rich media supporting growth of Lactobacilli species.

FIG. 5 depicts oleic acid selectively inhibiting three distinct strains of Gardnerella piotii, four distinct strains of Gardnerella vaginalis, and one strain of Gardnerella swindsiki-leopoldii.

FIG. 6 depicts genes encoding oleate hydratase enzyme (ohyA) and a putative fatty acid efflux pump (farE) (left) are uniquely absent in the pangenome of FGT L. iners while being nearly universally present in non-iners Lactobacilli genomes (right). In contrast, genes involved in the utilization of exogenous fatty acids (plsC, plsX, plsY) are universally present across all species, indicating a specific fatty acid response system absent in uniquely L. iners.Transcriptional profiling of L. crispatus treated with oleic acid in culture demonstrates upregulation of the oleate hydratase enzyme and putative fatty acid efflux pump, the two genes that are uniquely absent in L. iners (left).

FIG. 7 depicts oleic acid treatment alone and with Metronidazole inhibits Gardnerella piotii, Fannyhessea vaginae, and Sneathia amnii, including strains of G. piotii, F. vaginae, and S. amnii that are partially or wholly metronidazole-resistant.

FIG. 8 depicts oleic acid treatment alone and with Metronidazole shifting BV-like communities towards L. crispatus-dominance in rich, non-selective media in vitro.

FIG. 9 depicts co-culture of vaginal epithelial cells (VK2, ATCC) with a monoculture of each or mixture of L. crispatus and L. iners, showing that oleic acid substantially raises the ratio of L. crispatus to L. iners at concentrations that do not cause substantial human vaginal epithelial cell toxicity.

FIG. 10 depicts oleic acid (OA) or 10-hydroxystearic acid (10-HSA) treatment alone and with Metronidazole shifting BV-like communities towards L. crispatus-dominance in rich, non-selective media in vitro. Each bar represents a technical replicate per community condition. OA=Oleic acid at 3.17 mM (0.1% v/v), 10-HSA =10-hydroxystearic acid at 1.6 mM, MTZ=Metronidazole at 50 μg/mL.

FIG. 11 is a set of graphs showing that diverse unsaturated LCFAs inhibit L. iners and BV-associated species. LOA—Linoleic acid; LNA—Linolenic acid; DHA—cis-4,7,10,13,16,19-docosahexaenoic acid; ARA—arachidonic acid.

FIG. 12 depicts graphs showing the individual potency of diverse unsaturated LCFAs in L. crispatus growth promotion (left) and L. iners inhibition (right). EC2 is the concentration of the individual LCFAs to achieve a two-fold growth promotion in a lipid-poor media (modified MRS broth with lipids removed); the lower the EC2 indicates a more potent active ingredient. BC50 is the concentration of the individual or of the combined unsaturated LCFAs to achieve a two-fold growth reduction in a rich media (NYCIII broth); the lower the BC50 indicates a more potent active ingredient. VCA—cis-vaccenic acid; 10-HSA—10-hydroxystearic acid; ELA—Elaidic acid; OA—oleic acid; ARA—arachidonic acid; LNA—linolenic acid; LOA—linoleic acid; MOA—myristoleic acid; POA—palmitoleic acid; DHA—cis-4,7,10,13,16,19-docosahexaenoic acid; 10-HOA—10-hydroxy-12-octadecenoic acid.

FIG. 13 depicts graphs showing the combined effects of diverse unsaturated LCFAs on L. iners inhibition across different concentrations of each LCFA (50, 100, 200, and 400 μM) in a rich media (NYCIII broth). The growth of L. iners with each LCFA combination relative to its growth in media alone is shown by the depth of the shading. A darker shade indicates greater inhibition. VCA—cis-vaccenic acid; 10-HSA—10-hydroxystearic acid; ELA—Elaidic acid; OA—oleic acid; LNA—linolenic acid; MOA—myristoleic acid; POA—palmitoleic acid.

FIG. 14 depicts graphs showing the combined effects of diverse unsaturated LCFAs on L. crispatus growth promotion across different concentrations of each LCFA (50, 100, 200, and 400 μM) in a lipid-poor media (modified MRS broth with lipids removed). The growth of L. crispatus with each LCFA combination relative to its growth in media alone is shown by the depth of the shading. A darker shade indicates greater growth promotion. VCA—cis-vaccenic acid; 10-HSA—10-hydroxystearic acid; ELA—Elaidic acid; OA—oleic acid; LNA—linolenic acid; MOA—myristoleic acid; POA—palmitoleic acid.

FIG. 15 depicts graphs showing the individual and combined potency of diverse unsaturated LCFAs in L. crispatus growth promotion (left) and L. iners inhibition (right). EC2 is the concentration of the individual or of the combined unsaturated LCFAs to achieve a two-fold growth promotion in a lipid-poor media (modified MRS broth with lipids removed); the lower the EC2 indicates a more potent active ingredient. BC50 is the concentration of the individual or of the combined unsaturated LCFAs to achieve a two-fold growth reduction in a rich media (NYCIII broth); the lower the BC50 indicates a more potent active ingredient. The total concentration of LCFAs was held constant for an individual or combination of LCFAs tested. VCA—cis-vaccenic acid; 10-HSA—10-hydroxystearic acid; ELA—Elaidic acid; OA—oleic acid; ARA—arachidonic acid; LNA—linolenic acid; LOA—linoleic acid; MOA—myristoleic acid; POA—palmitoleic acid; DHA—cis-4,7,10,13,16,19-docosahexaenoic acid; 10-HOA—10-hydroxy-12-octadecenoic acid.

FIG. 16 depicts a set of graphs showing the individual and combined potency of diverse unsaturated LCFAs in the growth inhibition of diverse vaginal species. BC50 is the concentration of the individual or of the combined unsaturated LCFAs to achieve a two-fold growth reduction in a rich media (NYCIII broth). The figure depicts the BC50 by the depth of the shading; the lower the BC50 (darker shade) indicates a more potent active ingredient. Species shown include Candida albicans (C. albicans), Escherichia coli (E. coli), F. vaginae, G. piotii, G. vaginalis, Prevotella amnii (P. amnii), Prevotella bivia (P. bivia), Prevotella disiens (P. disiens), Prevotella timonensis (P. timonensis), S. amnii and Streptoccocus anginosus (S. anginosus). The total concentration of LCFAs was held constant for an individual or combination of LCFAs tested. VCA—cis-vaccenic acid; 10-HSA—10-hydroxystearic acid; ELA—Elaidic acid; OA—oleic acid; ARA—arachidonic acid; LNA—linolenic acid; LOA—linoleic acid; MOA—myristoleic acid; POA—palmitoleic acid; DHA—cis-4,7,10,13,16,19-docosahexaenoic acid; 10-HOA—10-hydroxy-12-octadecenoic acid.

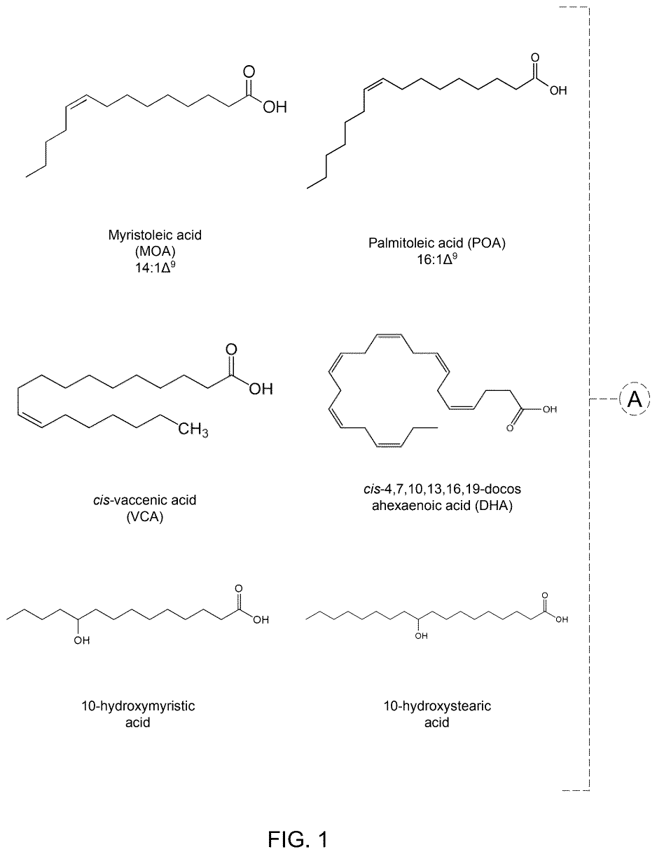

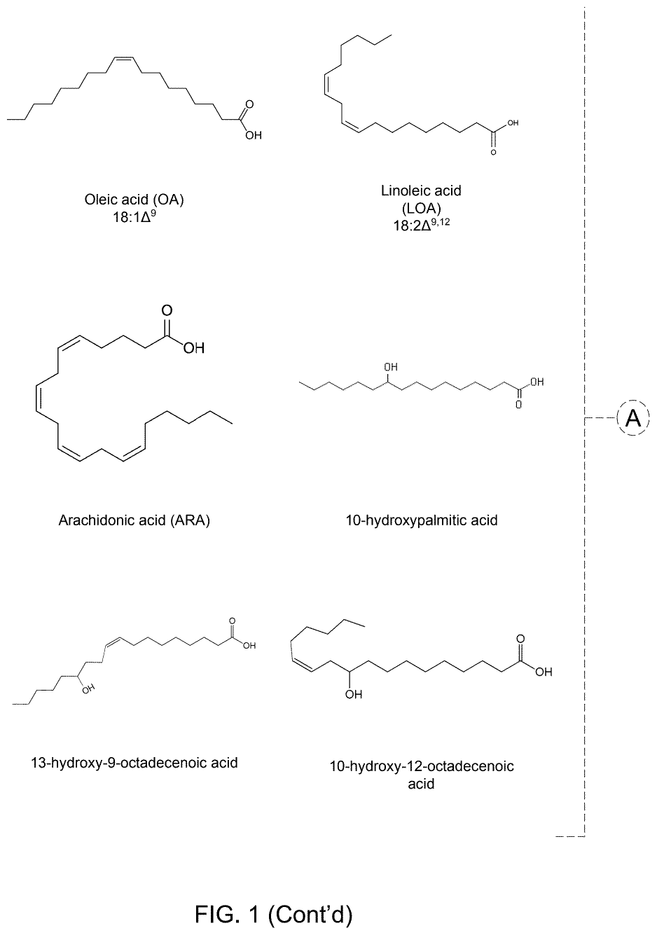

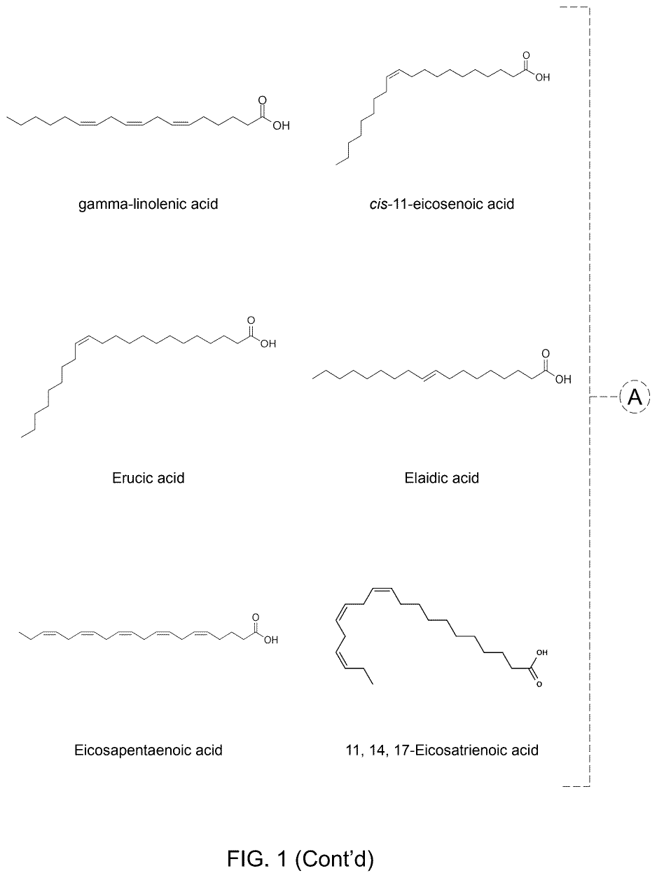

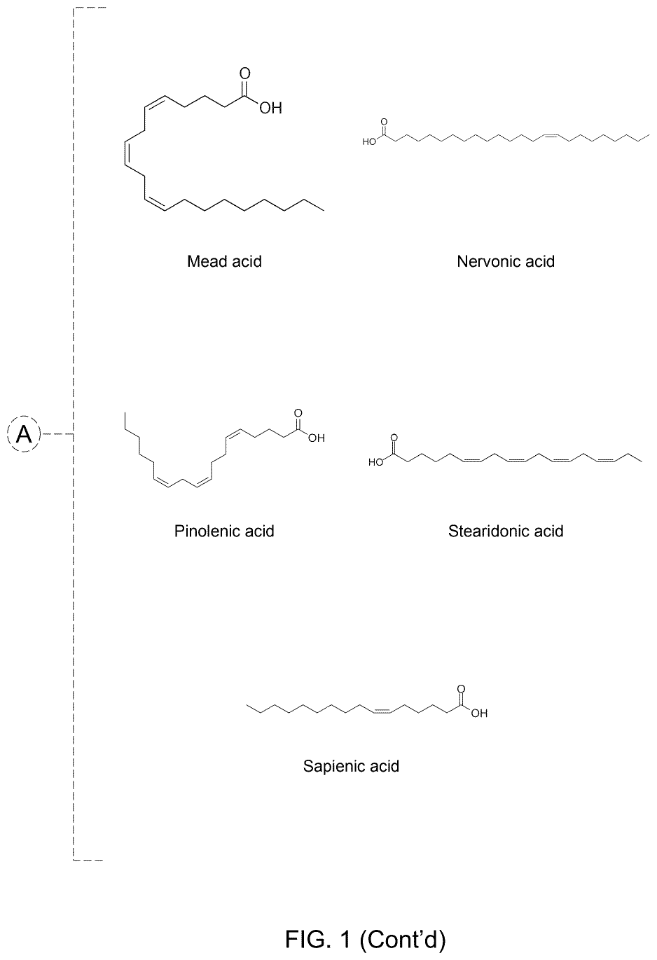

### DETAILED DESCRIPTION

### Definitions

Unless otherwise defined, all technical and scientific terms used herein have the same meaning as commonly understood by one of ordinary skill in the art to which this invention belongs. In case of conflict, the present application, including definitions will control.

A “individual” or “subject” is a vertebrate, including any member of the class Mammalia, including humans, domestic and farm animals, and zoo, sports or pet animals, such as mouse, rabbit, pig, sheep, goat, cattle and higher primates.

As used herein, the terms “treat,” “treating,” “treatment,” and the like refer to reducing or ameliorating vaginal dysbiosis, such as, a bacterial vaginosis disorder, and/or to ameliorate at least one symptom bacterial vaginosis (e.g., vaginal malodor, discharge, itching, irritation, or pain), and/or to ameliorate at least one symptom or aspect of disorders associated with bacterial vaginosis (e.g., cervical dysplasia, preterm birth, acquiring sexually transmitted infections, post-operative gynecological infections, or unsuccessful in vitro fertilization and spontaneous conception) in an individual in need thereof. It will be appreciated that, although not precluded, treating a disorder or condition does not require that the disorder, condition or symptoms associated therewith be completely eliminated.

BV is a vaginal dysbiosis disorder characterized by, for example, a depletion of Lactobacilli in the vaginal bacterial population, an increased diversity of the vaginal bacterial population, and an elevated vaginal pH (i.e., above about 4.5 pH). As used herein, “bacterial vaginosis” refers to a diagnosis of symptomatic or asymptomatic bacterial vaginosis that is diagnosed according to any one of a number of diagnostic tests or criteria familiar to one with ordinary skill in the art. These include the presence of at least 3 of 4 Amsel criteria (pH below or equal to 4.5, presence of clue cells on microscopic examination, a positive “whiff test” consisting of a “fishy” odor of vaginal discharge before or after addition of 10% potassium hydroxide, and presence of clue cells in vaginal fluid on microscopic examination), presence of a Gram stain-based Nugent score of greater than or equal to 7 or of greater than or equal to 4, a combination of these Amsel criteria and Nugent score results, or various other commercial diagnostic tests including, but not limited to, the Osom BV Blue test, the Affirm VP III test, the FemExam Test card, or other currently available tests or tests developed in the future that are validated for the diagnosis of bacterial vaginosis, or a combination of results from these tests accepted by one with ordinary skill in the art.

As used herein, “recurrent bacterial vaginosis” or “recurrence” or “relapse” refers to bacterial vaginosis diagnosed in a patient according to standard diagnostic tests or criteria 4 weeks or more after treatment of the same patient for a prior diagnosis of bacterial vaginosis.

In some embodiments, a subject in need of treatment may be diagnosed as having a relatively high abundance of L. iners with a paucity of L. crispatus. In some embodiments, the high abundance of L. iners results from administration of antimicrobial agents such as metronidazole or clindamycin. A subject in need of treatment may be experiencing a relapse of infection.

By “effective amount” or a “therapeutically effective amount” is meant the amount of unsaturated long chain fatty acids (U-LCFAs) and/or their metabolic derivatives thereof (also referred to herein as a hydroxylated derivative or a hydroxylated metabolic derivative thereof) that produce the desired therapeutic response (i.e., producing, restoring and/or enhancing vaginal function) and in specific embodiments, “effective amount” refers to the amount of U-LCFAs and/or their metabolic derivatives thereof needed to achieve vaginal colonization with a Lactobacillus-crispatus dominant microbial community, in which L. iners, Gardnerella spp., Fannyhessea vaginae, Sneathia amnii, and/or other relevant bacterial vaginosis-associated bacteria are selectively reduced or eradicated). Lactobacillus-crispatus dominant microbial communities resulting from “effective amounts” of U-LCFAs and/or their metabolic derivatives thereof may also include selectively increased levels of L. gasseri, L. jensenii, and/or L. mulieris.

References to hydroxylated fatty acids (e.g., 10-hydroxymyristic acid, 10-hydroxypalmitic acid, 10-hydroxystearic acid, 10-hydroxy-12-octadecenoic acid, and 13-hydroxy-9-octadecenoic acid or combinations thereof) can refer to molecules with S-configuration around each chiral carbon, R-configuration around each chiral carbon, or racemic mixtures consisting of a combination of molecules with S- and R-configurations around each chiral carbon.

Unless specifically stated or clear from context, as used herein, the term “about” is understood as within a range of normal tolerance in the art, for example within 2 standard deviations of the mean. “About” is understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

Other definitions appear in context throughout this disclosure.

### Compositions Comprising Unsaturated Long Chain Fatty Acids (U-LCFAs) and/or Their Metabolic Derivatives Thereof

Described herein are compositions and methods of treatment of vaginal dysbiosis (for example, bacterial vaginosis), or a disorder associated with vaginal dysbiosis (e.g., cervical dysplasia, reduction in the risk of preterm birth, reduction in the risk of acquiring sexually transmitted infections, reducing the risk of post-operative gynecological infections, or reducing the risk of unsuccessful in vitro fertilization and/or spontaneous conception) using vaginally administered U-LCFAs and/or their metabolic derivatives.

U-LCFAs as described herein comprise one, two or more (three or more, four or more, five or more, six or more, seven or more, eight or more, nine or more, ten or more, eleven or more, twelve or more, thirteen or more, fourteen or more, or fifteen or more) U-LCFAs, including but not limited to myristoleic acid, palmitoleic acid, oleic acid, linoleic acid,, cis-vaccenic acid, linolenic acid, gamma-linolenic acid, cis-11-eicosenoic acid, erucic acid, elaidic acid, eicosapentaenoic acid, 11,14,17-eicosatrienoic acid, mead acid, nervonic acid, pinolenic acid, stearidonic acid, sapienic acid, cis-4,7,10,13,16,19-docosahexaenoic acid, arachidonic acid, and each of their hydroxylated counterparts (e.g. 10-hydroxymyristic acid, 10-hydroxypalmitic acid, 10-hydroxystearic acid, 10-hydroxy-12-octadecenoic acid, and 13-hydroxy-9-octadecenoic acid).

The active ingredient consisting of one or more U-LCFAs or metabolic derivative thereof can be a combination which synergistically inhibits L. iners growth Synergistic combinations for inhibiting L. iners growth can comprise, or consist of elaidic acid and 10-hydroxystearic acid, elaidic acid and palmitoleic acid, elaidic acid and linolenic acid, elaidic acid and oleic acid, cis-vaccenic acid and palmitoleic, cis-vaccenic acid and oleic acid, and 10-hydroxystearic acid and 10-hydroxy-12-octadecenoic acid.

The active ingredient consisting of one or more U-LCFAs or metabolic derivative thereof can be a combination which synergistically increases L. crispatus growth. Synergistic combinations for increasing L. crispatus growth can comprise, or consist of cis-vaccenic acid and 10-hydroxystearic acid, elaidic acid and linolenic acid, 10-hydroxystearic acid and myristoleic acid, elaidic acid and palmitoleic acid, and elaidic acid and linolenic acid.

The active ingredient consisting of one or more U-LCFAs or metabolic derivative thereof can be a combination which inhibits diverse bacterial species contributing to bacterial vaginosis including elaidic acid and 10-hydroxystearic acid, cis-vaccenic acid and elaidic acid, and cis-vaccenic acid and 10-hydroxystearic acid. The diverse bacterial species contributing to bacterial vaginosis can include C. albicans, E. coli, F. vaginae, G. piotii, G. vaginalis P. amnii, P. bivia, P. disiens, P. timonensis, S. amnii, and S. anginosus.

The compositions comprising U-LCFAs and/or their metabolic derivatives thereof as described herein can be formulated for use as or in pharmaceutical compositions. Such compositions can be formulated or adapted for administration to a subject via any route, e.g., any route approved by the Food and Drug Administration (FDA). Exemplary methods are described in the FDA's CDER Data Standards Manual, version number 004 (which is available at fda.give/cder/dsm/DRG/drg00301.html).

In some embodiments, the active ingredient consists of one or more U-LCFAs and/or their metabolic derivatives; in other words, the one or more U-LCFAs and/or their metabolic derivatives thereof is/are the only active ingredient in the composition. By “active ingredient” it is meant that the one or more U-LCFAs and/or their metabolic derivatives thereof is/are the only chemical compound(s) that is eliciting a biological effect necessary to treat, prevent or reduce the risk of developing or recurrence, ameliorate, and/or mitigate vaginal dysbiosis (e.g., bacterial vaginosis) in a subject.

In some instances, the U-LCFAs and/or their metabolic derivatives thereof as described herein can be combined with pharmaceutical excipients known in the art to enhance preservation and maintenance of the U-LCFAs and/or their metabolic derivatives thereof or of bacterial isolates prior to administration to produce pharmaceutical compositions or pharmaceutical formulations. Any of the pharmaceutical composition, pharmaceutical formulations, or unit doses described herein can be administered to an individual in need thereof.

In some embodiments, pharmaceutical compositions including U-LCFAs and/or their metabolic derivatives thereof can also include any pharmaceutically acceptable carrier, adjuvant and/or vehicle. The term “pharmaceutically acceptable carrier or adjuvant” refers to a carrier or adjuvant that can be administered to a patient, together with a compound of this invention, and which does not destroy the pharmacological activity thereof and is nontoxic when administered in doses sufficient to deliver a therapeutic amount of the compound. As used herein the language “pharmaceutically acceptable carrier” includes saline, solvents, dispersion media, coatings, antibacterial and antifungal agents, isotonic and absorption delaying agents, and the like, compatible with pharmaceutical administration.

The pharmaceutical compositions can contain any conventional non-toxic pharmaceutically-acceptable carriers, adjuvants or vehicles. In some cases, the pH of the formulation can be adjusted with pharmaceutically acceptable acids, bases or buffers to enhance the stability of the formulated compound or its delivery form.

Pharmaceutical compositions are typically formulated to be compatible with its intended route of administration (e.g., oral, rectal parental, topical, suppository for vaginal administration, etc.). In some embodiments, U-LCFAs and/or their metabolic derivatives thereof compositions for vaginal administration can be formulated as suppositories (e.g., to be administered as suppositories). Exemplary suppositories can be prepared by mixing the particles with suitable non-irritating excipients or carriers such as cocoa butter, polyethylene glycol, or a suppository wax, which are solid at ambient temperature but liquid at body temperature and therefore melt in the vaginal cavity and release the U-LCFAs or their metabolic derivative (e.g., their hydroxylated derivative thereof).

In some embodiments, the U-LCFAs and/or their metabolic derivatives thereof can be formulated as a cream or gel for topical administration. For example, U-LCFAs and/or their metabolic derivatives thereof can be formulated as a vulvo-vestibular cream or gel. U-LCFAs and/or their metabolic derivatives thereof can be administered in a suspension in liquid form for use in a douche, in a capsule or vaginal tablet, or, for example, in dried form in a capsule or vaginal tablet.

In some embodiments the composition will be administered in a suspension in a vaginal capsule, or, for example, in dried form in a capsule. Compositions described herein can also be delivered via vaginal film. U-LCFAs and/or their metabolic derivatives thereof useful in the methods and compositions described herein can also be prepared and/or administered in admixture with one or more living biotherapeutic compositions that promote the maintenance, establishment and/or growth of the living biotherapeutic strains.

Therapeutic compositions containing the U-LCFAs and/or their metabolic derivatives thereof for the treatment of vaginal dysbiosis (e.g., bacterial vaginosis) can be conventionally administered in a unit dose. The term “unit dose” when used in reference to a therapeutic composition refers to physically discrete units suitable as unitary dosage for the individual, each unit containing a predetermined quantity of active material calculated to produce the desired therapeutic effect in association with the required physiologically acceptable diluent, i.e., carrier, or vehicle. Optimal dosing occurs over a duration of about two weeks or less.

U-LCFA and/or their metabolic derivatives thereof dosage forms, whether tablets, capsules, caplets, or particulates, may, if desired, be formulated so as to provide for controlled release of the therapeutic compositions, where controlled release maybe sustained release, delayed release, or a combination thereof. Controlled release formulations are preferably sustained release, meaning gradual delivery of the therapeutic compositions over an extended time period. Generally, as will be appreciated by those of ordinary skill in the art, sustained release dosage forms are formulated by dispersing the U-LCFAs and/or their metabolic derivatives thereof and other active agents/ingredients within a matrix of a gradually hydrolyzable material such as a hydrophilic polymer, or by coating a solid, drug-containing dosage form with such a material. Hydrophilic polymers useful for providing a sustained release coating or matrix include, by way of example: cellulosic polymers such as hydroxypropyl cellulose, hydroxyethyl cellulose, hydroxypropyl methyl cellulose, methyl cellulose, ethyl cellulose, cellulose acetate, and carboxymethylcellulose sodium; acrylic acid polymers and copolymers, preferably formed from acrylic acid, methacrylic acid, acrylic acid alkyl esters, methacrylic acid alkyl esters, and the like, e.g. copolymers of acrylic acid, methacrylic acid, methyl acrylate, ethyl acrylate, methyl methacrylate and/or ethyl methacrylate; and vinyl polymers and copolymers such as polyvinyl pyrrolidone, polyvinyl acetate, and ethylene-vinyl acetate copolymer.

In some embodiments, compositions as described herein can be conveniently provided as liquid preparations for vaginal, rectal, or oral administration, e.g., isotonic aqueous solutions, suspensions, emulsions, dispersions, or viscous compositions, which can be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Compositions described herein can be prepared as solutions. For example, solutions can be prepared by incorporating the U-LCFA and/or their metabolic derivatives thereof dosage forms, whether tablets, capsules, caplets, or particulates, may, if desired, be formulated so as to provide for controlled release of the therapeutic compositions, where controlled release maybe sustained release, delayed release, or a combination thereof utilized in the methods and compositions as described herein in the required amount of the appropriate solvent with various amounts of the other ingredients, as desired. Such compositions can be in admixture with a suitable carrier, diluent, or excipient such as sterile water, physiological saline, glucose, dextrose, or the like. The compositions can also be lyophilized.

The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, can be consulted to prepare suitable preparations, without undue experimentation.

Various additives which enhance the stability and sterility of the compositions, including antimicrobial preservatives, antioxidants, chelating agents, and buffers, can be added. Prevention or reducing risk of the action of microorganisms can be enabled or aided by various antibacterial and antifungal agents, for example, parabens, chlorobutanol, phenol, sorbic acid, and the like.

The compositions can be isotonic, i.e., the compositions can have the same osmolarity and/or osmolality as blood, lacrimal fluid, and cervicovaginal fluid. The desired isotonicity of the compositions as described herein can be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

Those skilled in the art will recognize that the components of the compositions should be selected to be chemically inert and will not impair the stability or efficacy of the U-LCFAs and/or their metabolic derivatives thereof described in the present disclosure. This will present no problem to those skilled in chemical and pharmaceutical principles, or problems can be readily avoided by reference to standard texts or by simple experiments (not involving undue experimentation), from this disclosure and the documents cited herein.

The skilled artisan can readily determine the amount of U-LCFA and/or their metabolic derivatives thereof dosage forms, whether tablets, capsules, caplets, or particulates, can, if desired, be formulated so as to provide for controlled release of the therapeutic compositions, where controlled release maybe sustained release, delayed release, or a combination thereof and optional additives, vehicles, and/or carrier in compositions and to be administered in methods as described herein. Of course, for any composition to be administered to a human, and for any particular method of administration, it is preferred to determine therefore: toxicity, the dosage of the composition(s), the concentration of components therein, and the timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

Therapeutic compositions disclosed herein can be formulated for sale in the US, imported into the US, and/or exported from the US. The pharmaceutical compositions can be included in a container, pack, or dispenser together with instructions for administration. In some embodiments, the invention provides kits that include the U-LCFAs and/or their metabolic derivatives thereof compounds. The kit may also include instructions for the physician and/or patient, suppository applicator, syringes, needles, box, bottles, vials, etc.

### Methods of Treatment

Contemplated herein are methods to treat individuals with L. iners-dominant microbiota to establish a microbiota dominated by non-iners lactobacilli.

Also contemplated herein are methods described herein can include methods for the treatment or reducing risk of vaginal dysbiosis (e.g., bacterial vaginosis) or vaginal dysbiosis (e.g., bacterial vaginosis) symptoms or having a disorder associated with vaginal dysbiosis (e.g., bacterial vaginosis) in an individual in need thereof using any of the compositions described herein.

Some embodiments are directed to methods for preventing or reducing the risk of recurrence (e.g., relapse) of vaginal dysbiosis (e.g., bacterial vaginosis), or symptoms of vaginal dysbiosis (e.g., bacterial vaginosis), or of a disorder associated with vaginal dysbiosis (e.g., bacterial vaginosis) (such as, cervical dysplasia, preterm birth, acquiring sexually transmitted infections, post-operative gynecological infections, or unsuccessful in vitro fertilization and spontaneous conception). Such methods of preventing or reducing the risk of recurrence of vaginal dysbiosis (e.g., bacterial vaginosis), symptoms of vaginal dysbiosis (e.g., bacterial vaginosis), or a disorder associated with vaginal dysbiosis (e.g., bacterial vaginosis) (such as, cervical dysplasia, preterm birth, acquiring sexually transmitted infections, post-operative gynecological infections, or unsuccessful in vitro fertilization and spontaneous conception) can comprise administering to a subject in need thereof any of the compositions described herein following, during, or prior to administration of an antimicrobial agent, including antimicrobial agents known to be susceptible to relapse, such as metronidazole and clindamycin, to the subject, and/or following, during, or prior to administration of a biotherapeutic product such as a live biotherapeutic (LBP, e.g., a probiotic) containing one or more strains of bacteria to the subject intended to treat vaginal dysbiosis (e.g., bacterial vaginosis), prevent vaginal dysbiosis (e.g., bacterial vaginosis), prevent recurrent vaginal dysbiosis (e.g., bacterial vaginosis), or establish a healthy vaginal microbiota, and/or following, during, or prior to administration of a vaginal microbiota transplant to the subject intended to treat bacterial vaginosis, prevent bacterial vaginosis, prevent recurrent bacterial vaginosis, or establish a healthy vaginal microbiota.

In any of the methods describe above, the concentration of the one or more U-LCFAs and/or their metabolic derivatives thereof in the compositions described herein is about 0.01-30% v/v (e.g., about 0.1% v/v, about 0.2% v/v, about 0.3% v/v, about 0.4% v/v, about 0.5% v/v, about 0.6% v/v, about 0.7% v/v, about 0.8% v/v, about 0.9% v/v, about 1.0% v/v, about 2.0% v/v, about 3.0% v/v, about 4.0% v/v, about 5.0% v/v, about 6.0% v/v, about 7.0% v/v, about 8.0% v/v, about 9.0% v/v, about 10.0% v/v, about 11.0% v/v, about 12.0% v/v, about 13.0% v/v, about 14.0% v/v, about 15.0% v/v, about 16.0% v/v, about 17.0% v/v, about 18.0% v/v, about 19.0% v/v, about 20.0% v/v, about 21.0% v/v, about 22.0% v/v, about 23.0% v/v, about 24.0% v/v, about 25.0% v/v, about 26.0% v/v, about 27.0% v/v, about 28.0% v/v, about 29.0% v/v, about 30.0% v/v or any increment in between).

In some embodiments, the composition comprising the one or more U-LCFAs and/or their metabolic derivatives thereof can be administered daily, weekly, or monthly. In some embodiments, the composition is administered once a day, twice a day, or three times a day or more. The compounds can be administered over a period of weeks, months, or years. For example, in some embodiments, the composition can be administered once or twice a day for a period of at least about 1 week, 2 weeks, 3 weeks, 1 month, 2 months, 3 months, 4 months, 5 months, 6 months, 7 months, 8 months, 9 months, 10 months, 11 months, 1 year, 2 years, 3 years, 4 years, or at least or about 5 years, or more.

In some embodiments, the subject in need thereof was previously or is presently receiving an antimicrobial agent, such as metronidazole and clindamycin. In some embodiments, the subject has not received an antimicrobial agent, such as metronidazole and clindamycin. In some embodiments, the subject is on a first regimen, wherein the first regimen comprises administration of a composition comprising one or more U-LCFAs and/or their metabolic derivatives thereof and then receiving an antimicrobial agent, such as metronidazole and clindamycin (wherein the antimicrobial agent can be administered alone or in conjunction with the composition comprising one or more U-LCFAs and/or their metabolic derivatives thereof).

In some embodiments, the subject in need thereof was previously or is presently receiving a live biotherapeutic (LBP). As described herein, in some instances LBP and probiotic are used interchangeably and refer to the same thing. In some embodiments, the subject has not received a LBP. In some embodiments, the subject is on a first regimen, wherein the first regimen comprises administration of a composition comprising one or more U-LCFAs and/or their metabolic derivatives thereof and then receiving a LBP (wherein the antimicrobial agent can be administered alone or in conjunction with the composition comprising one or more U-LCFAs and/or their metabolic derivatives thereof). LBPs contemplated herein include such as those containing one or more of Lactobacillus crispatus, Lactobacillus gasseri, Lactobacillus jensenii, or Lactobacillus mulieris. Other LBPs contemplated herein are described in PCT/US2023/063490, which is incorporated herein by reference. For example, the LBP regimen includes the LC-106 LBP composition comprising or consisting of L. crispatus strains C0022A1, C0175A1, C0059E1, FF00051, FF00018 and UC101_2_33, the LC-115 LBP composition comprising or consisting of L. crispatus strains C0022A1, C0112A1, C0175A1, C0059E1, C0028A1, C0006A1, UC101_2_33, UC119_2_11, FF00004, FF00018, FF00064, FF00051, FF00072, 185329_1999_17 and 122010_1999_16

For example, a subject undergoing a relapse or recurrence of vaginal dysbiosis (e.g., bacterial vaginosis) after prior antimicrobial and/or live biotherapeutic (e.g., a probiotic) treatment can repeat antimicrobial or live biotherapeutic treatment together with treatment of U-LCFAs and/or their metabolic derivatives thereof. Antimicrobial agents, such as metronidazole can be administered vaginally (e.g., vaginal metronidazole 0.75% gel; a 5-gram applicator is inserted into the vagina once daily for five days) or orally (e.g., oral metronidazole 500 mg taken twice daily for seven days). In addition, vaginal clindamycin cream (e.g., at a concentration of 2%) can be given vaginally as 5 grams of cream daily for seven days. In some embodiments, probiotic or live biotherapeutic agents can be administered intravaginally (for example the L. crispatus-containing product LACTIN-V) or orally.

The methods of treating vaginal dysbiosis (e.g., bacterial vaginosis) and/or Lactobacillus iners-dominant vaginal colonization can include an alleviation of one or more symptoms of vaginal dysbiosis (e.g., bacterial vaginosis) and/or Lactobacillus iners-dominant vaginal colonization. For example, a method of treating vaginal dysbiosis (e.g., bacterial vaginosis) and/or Lactobacillus iners-dominant vaginal colonization comprises or further comprises an alleviation of one or more symptoms of vaginal dysbiosis (e.g., bacterial vaginosis) and/or Lactobacillus iners-dominant vaginal colonization. The one or more symptoms can include, but are not limited to, abnormal vaginal odor, abnormal vaginal discharge, vulvovaginal itching, discomfort with sexual intercourse, or a combination thereof. “Alleviation” refers to a lessening of the severity of one or more symptoms.

Any of the methods or compositions described herein can also include a cysteine and/or cystine uptake inhibitor. For example, a cysteine and/or cystine uptake inhibitor can prevent a bacterial cell (e.g., a cell of a bacterial species contributing to vaginal dysbiosis (e.g., bacterial vaginosis)), from transporting cysteine and/or cystine into the cell from outside the cell. A cysteine and/or cystine uptake inhibitor can also or alternatively selectively inhibit Lactobacillus iners growth in vitro or in vivo. Non-limiting cysteine and/or cystine uptake inhibitors can include S-methyl-L-cysteine (SMC) and seleno-DL-cysteine (SDLC), and related compounds or compounds exerting a similar effect. (See, for example, Bloom S M et al. Nat. Microbiol., (2022) Mar;7(3):434-450, which is incorporated in its entirety.)

### Subject Selection/Diagnosis

### Lactobacillus iners-Dominant Vaginal Colonization

In some embodiments, a subject in need of treatment can be diagnosed as having a relatively high abundance (i.e., >50% relative abundance) of L. iners with a depletion of L. crispatus. Vaginal microbiome composition may be determined by 16s rRNA gene sequencing from a vaginal sample. Standard testing is available from companies that provide this service, e.g. Evvy vaginal health tests and Juno Bio.

The methods described herein can be used for restoring Lactobacillus dominated microbiota in a subject in need thereof. Such methods of restoring Lactobacillus dominated microbiota can comprise administering to a subject in need thereof any of the compositions described herein (e.g., those comprising one or more U-LCFAs and/or their metabolic derivatives thereof). The individual can have low Lactobacillus prevalence or no Lactobacillus in their vaginal microbiota. The subject may have undergone metronidazole and/or clindamycin treatment. In some embodiments, U-LCFAs and/or their metabolic derivatives thereof achieve vaginal colonization with a Lactobacillus-crispatus dominant microbial community by selective reduction or eradication of L. iners, Gardnerella spp., Fannyhessea vaginae, Sneathia amnii, and/or other bacterial vaginosis-associated bacteria. Lactobacillus-crispatus dominant microbial communities resulting from treatments including one or more U-LCFAs and/or their metabolic derivatives thereof may also include selectively increased levels of L. gasseri, L. jensenii, and/or L. mulieris.

### Vaginal Dysbiosis

In some embodiments, a subject in need of treatment can be diagnosed as having a relatively low abundance (i.e., <50% relative abundance) of non-iners Lactobacillus.

In some embodiments, the individual is an otherwise healthy individual. In some embodiments, the individual is a pregnant female. In some embodiments, the individual is an otherwise healthy pregnant female. In some embodiments, the individual is an individual having had 3 or fewer vaginal dysbiosis (e.g., bacterial vaginosis) infections in the past 12 months. In some embodiments, the individual is an individual having had 3 or more vaginal dysbiosis (e.g., bacterial vaginosis) infections in the past twelve months (or two in a six month period). In some embodiments, the individual is an individual with recurring vaginal dysbiosis (e.g., bacterial vaginosis). In some embodiments, the individual is an individual with frequent vaginal dysbiosis (e.g., bacterial vaginosis) infections. In some embodiments, the individual is an individual with a first-time episode of vaginal dysbiosis (e.g., bacterial vaginosis). Administration of a therapeutically effective amount of any of the compositions described herein for the treatment of any of the conditions described herein associated with vaginal dysbiosis (e.g., bacterial vaginosis) can result in a reduction in the amount or severity of vaginal discharge with foul smelling odor, a reduction in the amount of grayish or white vaginal discharge, a reduction in a burning sensation that can occur while urinating, a reduction in discomfort with sexual intercourse, or a reduction in itchiness around the vaginal area, inter alia.

Vaginal dysbiosis (e.g., bacterial vaginosis) can be identified using a variety of mechanisms. For example, vaginal dysbiosis (e.g., bacterial vaginosis) can be identified using either the Nugent scoring system or the Amsel criteria. The Nugent scoring system (as described in journals.asm.org/doi/10.1128/jcm.29.2.297-301.1991) uses vaginal smears that are plated on a microscopic slide in oil immersion, and a minimum of 10 high power fields are examined for three bacteria morphotypes: Lactobacillus, Gardnerella, and curved gram-variable rods. Each of these three categories receives a score based on the number of bacteria counted. Subsequently, these three scores are added together for a total score ranging from 0-10. A score of 0-3 is negative for vaginal dysbiosis (e.g., bacterial vaginosis), a score of 4-6 is intermediate for vaginal dysbiosis (e.g., bacterial vaginosis), and a score of 7+ is positive for vaginal dysbiosis (e.g., bacterial vaginosis).

Alternatively, the Amsel criteria can be used. There are four parameters used to determine the presence or absence of vaginal dysbiosis (e.g., bacterial vaginosis). These are 1) thin, white, yellow, homogeneous discharge, 2) clue cells on wet mount microscopy, 3) a vaginal fluid pH greater than 4.5 when placing the discharge on litmus paper, and 4) a release fishy odor after adding 10% potassium hydroxide (KOH) solution to wet mount (also known as “whiff test”). A clue cell is a type of vaginal epithelial cell to which a cluster of bacteria is attached. At least two of the four symptoms are present to receive a positive vaginal dysbiosis (e.g., bacterial vaginosis) test result. Preferably, at least three of the four symptoms of the Amsel criteria are present.

Vaginal dysbiosis (e.g., bacterial vaginosis) can also be identified or diagnosed using molecular techniques, and can be called a molecular diagnosis. A molecular diagnosis of vaginal dysbiosis (e.g., bacterial vaginosis) identifies or diagnoses vaginal dysbiosis (e.g., bacterial vaginosis)-like states using bacterial sequencing methods.

Vaginal dysbiosis (e.g., bacterial vaginosis) can also be identified using various clinical diagnostic options such as an enzyme-based tests (e.g., OSOM® BVBlue), DNA probe tests (e.g., BD Affirm™ VPIII), or multi-target PCR tests (e.g., BD Max™, GeneXpert®, or Aptima® BV assay).

Vaginal dysbiosis (e.g., bacterial vaginosis) can also be identified or diagnosed in some embodiments via an accepted combination of the above diagnostic criteria.

In some embodiments, the individual is a female presenting with an off-white (e.g., milky or gray), thin, homogeneous vaginal discharge, vaginal pH greater than or equal to 4.7, presence of clue cells of greater than or equal to 20% of the total epithelial cells on microscopic examination of a vaginal saline wet mount, a positive 10% KOH whiff test or a combination thereof. In some embodiments, the individual is a female presenting with an off-white (milky or gray), thin, homogeneous vaginal discharge, vaginal pH greater than or equal to 4.7, the presence of clue cells of greater than or equal to 20% of the total epithelial cells on microscopic examination of a vaginal saline wet mount, and a positive 10% KOH whiff test. In some embodiments, the individual is a female with confirmed vaginal dysbiosis (e.g., bacterial vaginosis). In some embodiments, vaginal dysbiosis (e.g., bacterial vaginosis) is confirmed by the presence of three (3) or four (4) Amsel criteria parameters selected from an off-white (milky or gray), thin, homogeneous vaginal discharge, vaginal pH greater than or equal to 4.7, the presence of clue cells of greater than or equal to 20% of the total epithelial cells on microscopic examination of a vaginal saline wet mount, a positive 10% KOH Whiff test and a gram stain slide Nugent score equal to, or higher than four (4) on bacterial analysis of vaginal samples. In some embodiments, the individual is a female with suspected vaginal dysbiosis (e.g., bacterial vaginosis). In some embodiments, suspected vaginal dysbiosis (e.g., bacterial vaginosis) is indicated by the presence of three (3) or four (4) Amsel criteria parameters selected from an off-white (milky or gray), thin, homogeneous vaginal discharge, vaginal pH greater than or equal to 4.7, the presence of clue cells of greater than or equal to 20%, of the total epithelial cells on microscopic examination of a vaginal saline wet mount, and a positive 10% KOH Whiff test. In some embodiments, the individual is a female presenting with an off-white (milky or gray), thin, homogeneous vaginal discharge, odor, or a combination thereof.

The present invention is additionally described by way of the following illustrative, non-limiting Examples that provide a better understanding of the present invention and of its many advantages.

### EXAMPLES

The invention is further described in the following examples, which do not limit the scope of the invention described in the claims.

### Materials and Methods

The following materials and methods were used to conduct the experiments described herein.

Bacterial liquid and solid culture media: Bacteria were cultured under anaerobic conditions at 37-40° C. using a palladium catalyst-based anaerobic chamber (COY) with an atmosphere of 5% carbon dioxide, 5% hydrogen, and 90% nitrogen. All media, culture reagents, and plastic-ware were pre-reduced by placement overnight in the anaerobic chamber before use. Media additives were added to autoclave-sterilized broth media or agar media after cooling to room temperature. Broth media was re-sterilized using a 0.22 uM vacuum filter after all additives were added. All cultured bacterial isolates were first revived on solid media agar to obtain single colonies. After 4-5 days of anaerobic incubation, representative colonies were picked into liquid culture. All Lactobacillus species isolates were revived from frozen 25% glycerol stocks on Hardy MRS agar, except for Lactobacillus iners, which were revived on Hardy Columbia blood (CBA) agar. Non-Lactobacillus species were revived from frozen 25% glycerol stocks on Hardy CBA agar. For cultivation of Lactobacillus species in liquid media, MRS broth media with cysteine and glutamine (MRSQC) was used unless otherwise noted. For cultivation of non-Lactobacillus species, NYCIII broth was used unless otherwise noted. To prepare MRSQC, BD Difco-formulated De Man, Rogosa and Sharpe (MRS) broth powder was prepared as per the manufacturer's instructions and then supplemented with L-cysteine (4 mM) and L. glutamine (1.1 mM). For long chain fatty acid additives, MOA, POA, OA, and LOA were purchased from Sigma as ≥98.5% (GC) purity. NYCIII broth media (American Type Culture Collection (ATCC) medium 1685) pre-media is prepared with 15 g/L proteose peptone no. 3, 5 g/L sodium chloride, and 4 g/L HEPES in 875 mL of distilled water, pH-adjusted to 7.3, and autoclaved. After autoclaving and cooling the pre-media, dextrose solution (3 g/45 mL distilled water) is added at 7.5% v/v, Gibco yeast extract solution is added at 2.5% v/v, and heat inactivated horse serum is added at 10% v/v. The complete NYCIII broth is then sterilized by passage through a 0.22 μm vacuum filter. S-broth pre-media is prepared with 37 g/L brian heart infusion broth, 10 g/L yeast extract powder, and 1 g/L dextrose in 880 mL distilled water, brought to a boil until dissolved, and then autoclaved. After autoclaving and cooling the pre-media, fetal bovine serum is added at 5% v/v, vitamin K1-Hemin solution is added at 5% v/v, and IsoVitaleX enrichment is added at 2% v/v. The complete S-broth is then sterilized by passage through a 0.22 μm vacuum filter. Unless otherwise indicated, S-broth was also supplemented with 1 g/L Tween 80 before the vacuum filter step.

Bacterial culture conditions and media additives for growth inhibition, growth enhancement, and killing assays: To assess monoculture growth effects, bacterial isolates were first revived on solid media for 2-5 days until fully formed colonies were present. Starter liquid cultures were then inoculated with representative colonies for each strain and grown for 48 hours. After 48 hours, the optical density at 600 nm (OD600) of each liquid monoculture was measured and each cultivated strain was back-diluted to the starting OD (OD=0.02) for each experiment using the base media specific to that experiment. In cases where growth promotion was being assessed, starter cultures were pelleted (5,000×g, 5 min, 4° C.) and washed with phosphate-buffered saline (PBS) three times and resuspended in PBS prior to experiment inoculation to prevent nutrient carryover. Data from growth experiments are presented as the median +/− range for three technical replicates. Figures depict representative results from one of two or more independent experiments with distinct batches of freshly prepared media and input bacterial inocula for each strain and media condition.

Competition and mock community culture experiments: Starter cultures of representative strains of L. crispatus, L. gasseri, L. iners, L. jensenii, G. piotii, G. vaginalis, P. amnii, P. bivia, P. disiens, P. timonensis, F. vaginae, and S. amnii were prepared in NYCIII broth media. Aliquots of the axenic cultures were mixed in defined ratios and then divided into replicate cultures across different treatment conditions for incubation (6 replicates per condition) by adding 150 μL of mixture in V-bottom 96 well plates. Treatment conditions included NYCIII untreated, NYCIII+3.17 mM oleic acid, NYCIII+50 μg/mL metronidazole, and NYCIII+3.17 mM oleic acid+50 μg/mL metronidazole. C.F.U. titers were determined for each axenic culture as described above and used to calculate starting ratios within the mixed cultures. At 24, 48, and 72 hours, cultures were harvested by centrifuging at 5,000×g for 10 min at 4° C., supernatant was removed, and pellets were frozen for later DNA extraction and analysis. Relative growth within mixed cultures was assessed by bacterial 16S rRNA gene sequencing as described below. C.F.U. titers were determined for each community mixture at the t=0 hrs and the final time point to confirm growth of the defined, in vitro community cultures. Aliquots of axenic cultures were assayed separately in all treatment conditions to confirm expected growth patterns, and assessed by OD600 at the same corresponding time points.

Cross-culture experiments and LDH assay: Starter cultures of L. crispatus and L. iners were prepared in MRSQC broth media. Cultures were pelleted (5,000×g, 5 min, 4° C.) and washed with phosphate-buffered saline (PBS) three times and resuspended in PBS prior to experiment inoculation to prevent nutrient carryover. Cultures were resuspended to an OD=0.8 in PBS. A mixture of L. crispatus and L. iners was prepared by making a 1:10 ratio mixture of the L. crispatus:L. iners cultures. Each axenic and mixture culture was used to inoculate plates seeded with the vaginal epithelial cell line, VK2., in kSFM +/− oleic acid. VK2 cells with live bacteria were incubated for 24 hours anaerobically at 37 C. Cytotoxicity of the VK2 cells were measured using an LDH release cytotoxicity assay following the manufacturer's protocol. To determine the bacterial CFU titre, we used a selective plating method to infer relative titres of L. crispatus and L. iners. MRS agar only supports the growth of L. crispatus, and therefore was used to measure the bacterial titre of L. crispatus only. CBA agar supports the growth of both L. crispatus and L. iners, and therefore was used to measure the bacterial titre of the total community. Thus, for the mixed cultures, the number of colonies recovered on MRS relative to the number of colonies recovered on CBA agar represented the proportion of L. crispatus in the mixed culture.

### Example 1. Growth Inhibition of L. Iners by U-LCFAs

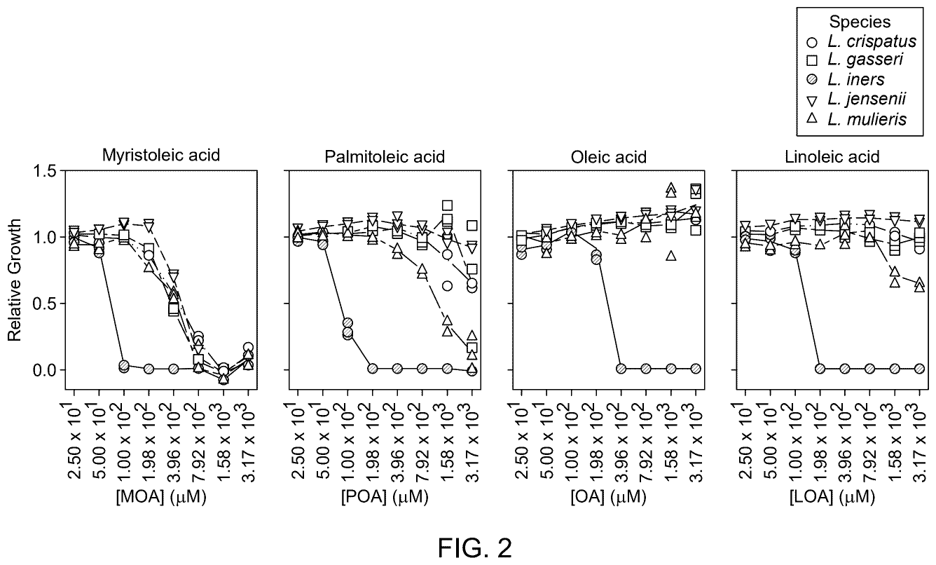

U-LCFAs (myristoleic acid, palmitoleic acid, oleic acid, and linoleic acid) inhibited three representative strains of L. iners at significantly lower concentrations compared to representative strains of L. crispatus, L. gasseri, L. jensenii, and L. mulieris (FIG. 2). Growth was assessed by optical density at 600 nm (OD₆₀₀) and plotted as median ±range for 3 replicates from 1 of ≥2 independent experiments per strain and media condition. All strains were cultured in Difco MRS broth with 4 mM L-cysteine and 1.1 mM L-glutamine.

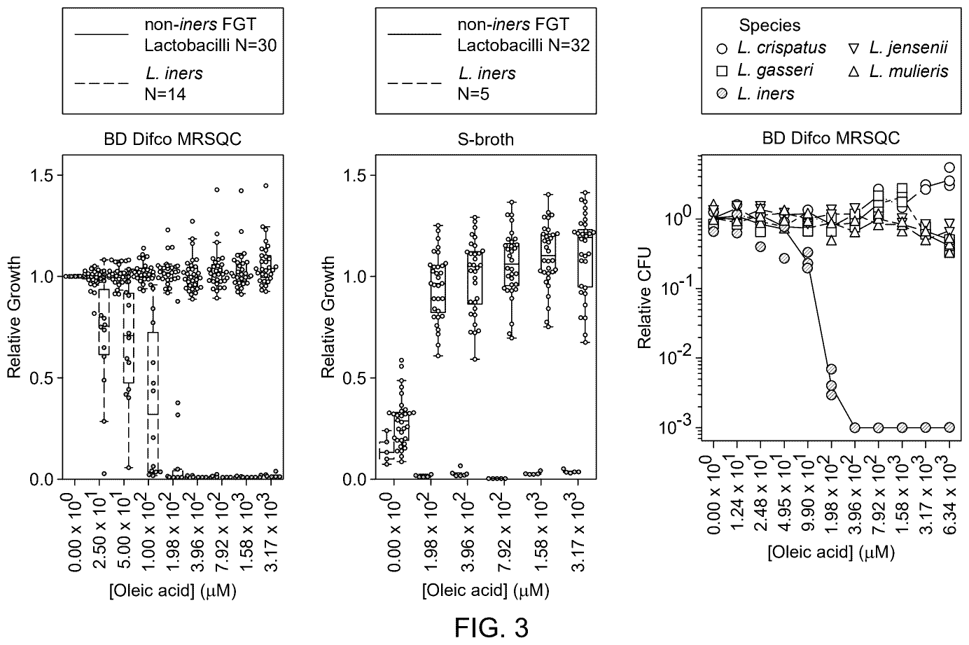

Oleic acid selectively inhibited 14 distinct strains of L. iners compared to 30 distinct strains of L. crispatus, L. gasseri, L. jensenii, and L. mulieris in rich media supporting growth of Lactobacilli species (FIG. 3, left). Oleic acid selectively enhanced growth of 32 distinct strains of non-iners Lactobacilli in nutrient-limited media (FIG. 3, middle). Growth was assessed by optical density at 600 nm (OD₆₀₀) and plotted as median ±range for 3 replicates from 1 of ≥2 independent experiments per strain and media condition.

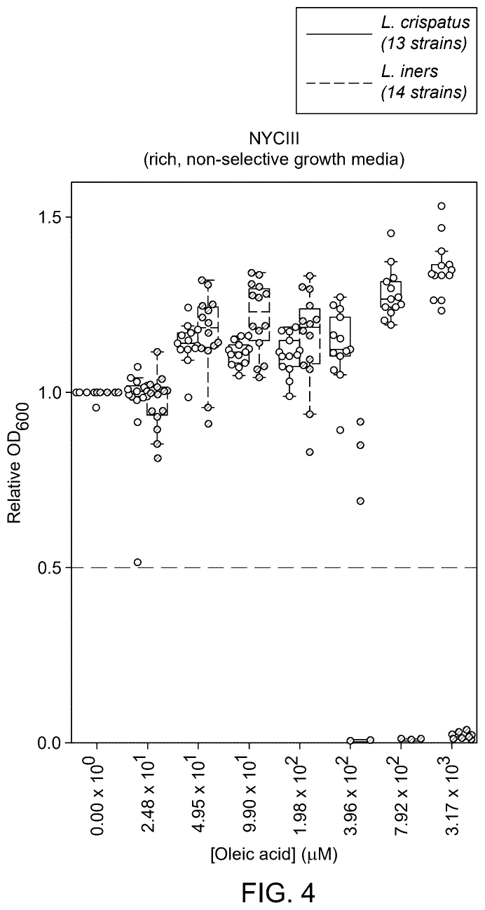

Oleic acid selectively inhibited 14 distinct strains of L. iners (FIG. 4, left) compared to 13 distinct strains of L. crispatus, L. gasseri, L. jensenii, and L. mulieris in serum-based rich media supporting growth of Lactobacilli species (FIG. 4, right).

Oleic acid was selectively bactericidal towards two representative strains of L. iners compared to representative strains of non-iners Lactobacilli. The inhibitory effect of oleic acid against L. iners was observed to be bactericidal (FIG. 3, right). Colony forming units (CFU) were counted from 24-hr treated cells in liquid culture spotted onto Hardy Columbia blood agar for L. iners and MRS agar plates for non-iners Lactobacilli. The median ± range for 3 biological replicates per strain and media condition are plotted from 1 of ≥2 independent experiments.

### Example 2. Oleic Acid Selectively Inhibits Other Disease-Associated FGT Species

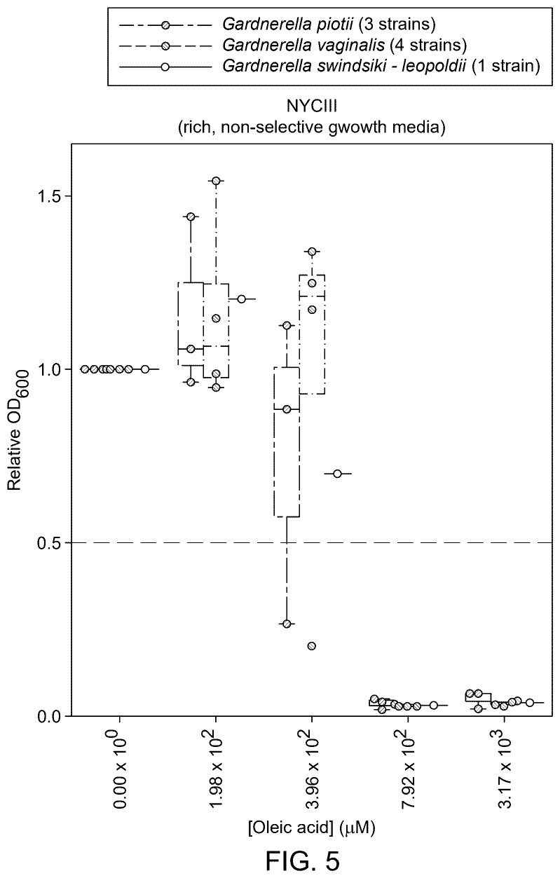

Oleic acid selectively inhibited three distinct strains of G. piotii, four distinct strains of G. vaginalis, and one strain of G. swindsiki-leopoldii (FIG. 5). Growth was assessed by optical density at 600 nm (OD₆₀₀) and plotted as median ± range for 3 replicates from 1 of ≥2 independent experiments per strain and media condition.

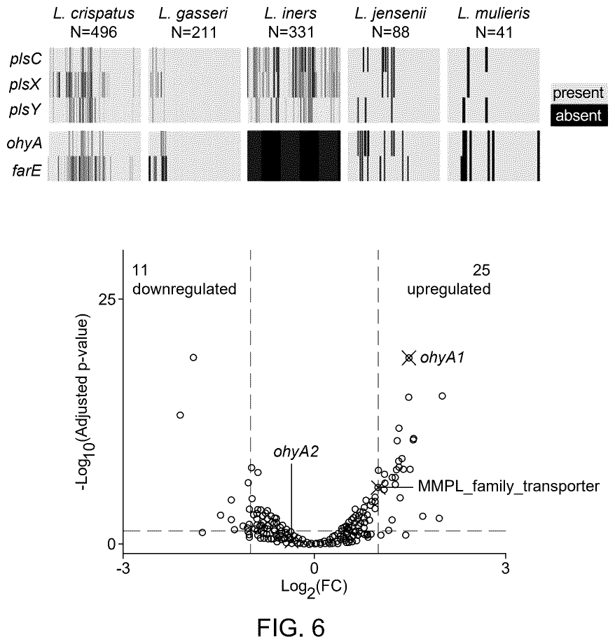

Genes encoding for oleate hydratase enzyme (ohyA) and a putative fatty acid efflux pump (farE) (FIG. 6, left) are uniquely absent in the pangenome of FGT L. iners while being nearly universally present in non-iners Lactobacilli genomes (FIG. 6, right). Transcriptional profiling in L. crispatus show that an oleate hydratase (ohyA) gene and putative fatty acid efflux pump (MMPL_family_transporter) are upregulated by oleic acid treatment.

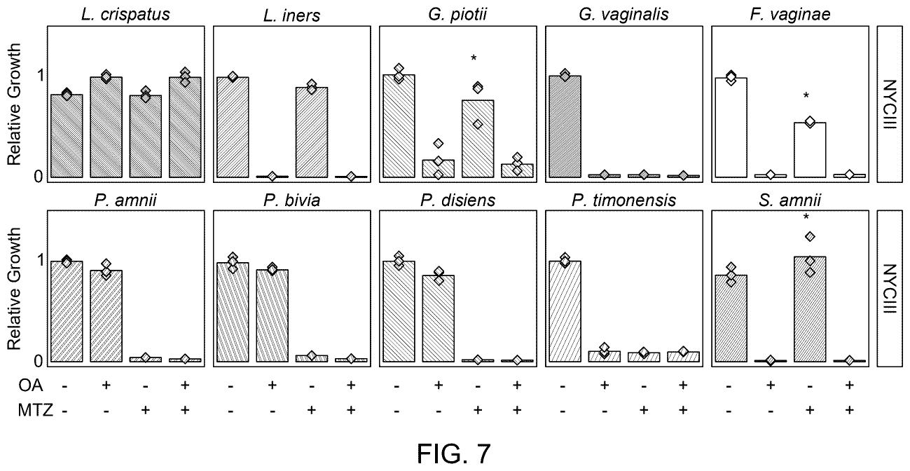

Oleic acid treatment alone and with Metronidazole inhibits other disease-associated FGT species, including Metronidazole-resistant strains of Gardnerella piotii, Fannyhessea vaginae, and Sneathia amnii (FIG. 7). Growth was assessed by optical density at 600 nm (OD₆₀₀) at 72 hours. Relative growth was calculated as the OD₆₀₀ relative to the condition exhibiting maximal growth per strain. Each point represents a technical replicate for a single strain and condition. OA=Oleic acid at 3.17 mM (0.1% v/v), MTZ=Metronidazole at 50 μg/mL.

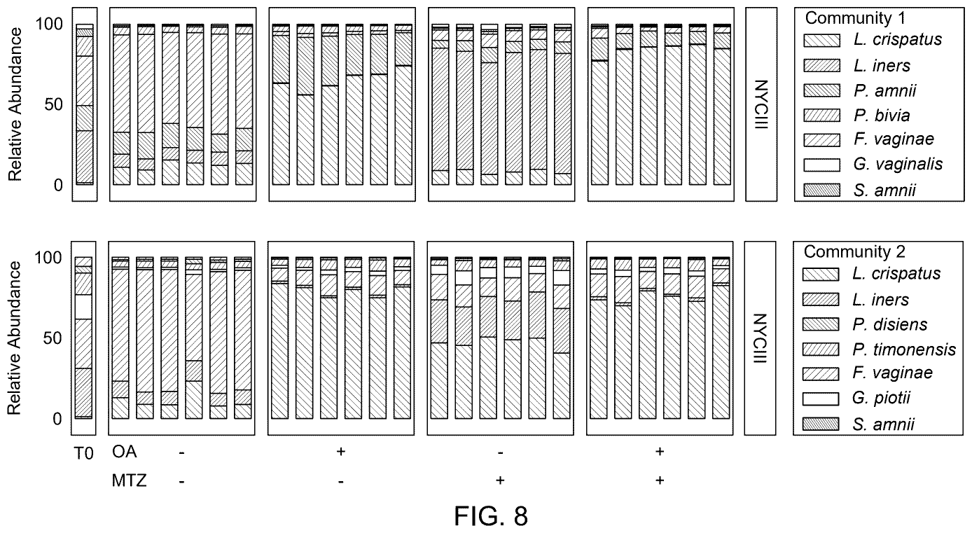

Oleic acid treatment alone and with Metronidazole shifted BV-like communities towards L. crispatus-dominance in rich, non-selective media in vitro (FIG. 8). 16s rRNA gene sequencing was performed on samples collected at 0 and 72 hours to determine community taxonomy composition. Each bar represents a technical replicate per community condition. OA=Oleic acid at 3.17 mM (0.1% v/v), MTZ=Metronidazole at 50 μg/mL.

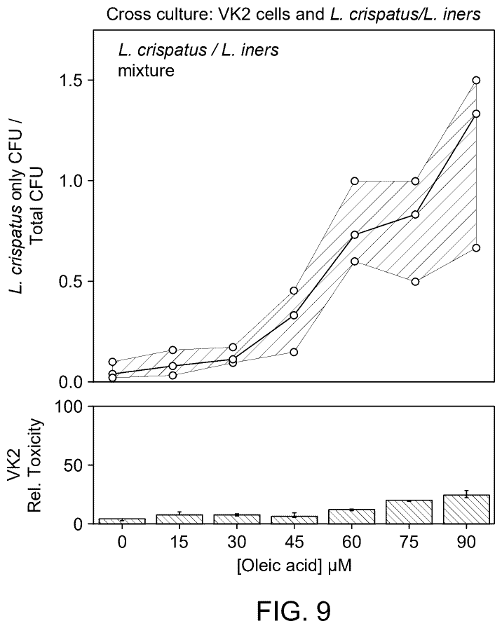

Co-culture of vaginal epithelial cells (VK2, ATCC) with a monoculture of each or mixture of L. crispatus and L. iners demonstrated that oleic acid can be used to shift a two-species community of L. crispatus and L. iners from L. iners dominance to L. crispatus dominance without exhibiting high significant levels of toxicity to the vaginal epithelial cells (FIG. 9). VK2 relative toxicity was measured using a commercial LDH release assay and CFU of L. crispatus and L. iners is measured by colony counting on species-selective agar.

### Example 3. 10-HSA Selectively Promotes L. crispatus Growth In Vivo

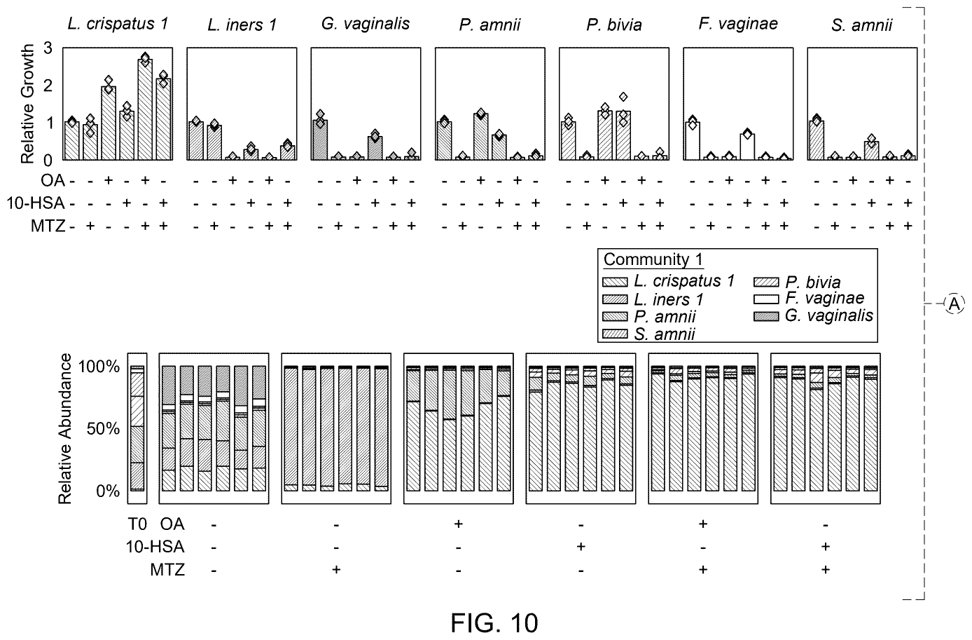

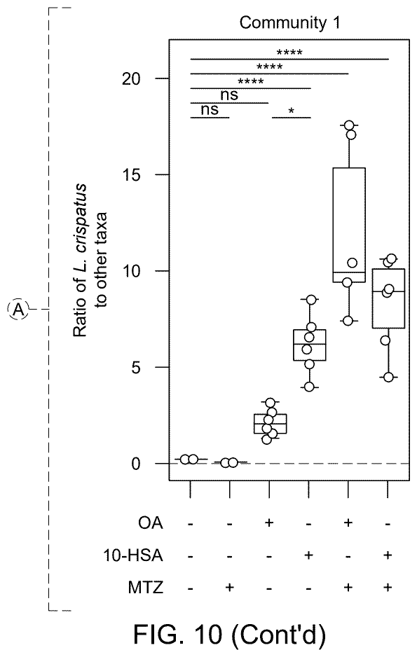

In vitro BV-like community competition assays were performed with 1.6 mM 10-HSA treatment (10-HSA is the metabolic derivative of oleic acid) in addition to 3.17 mM oleic acid treatment, each alone or in combination with 50 μg/mL metronidazole (MTZ). 16s rRNA gene sequencing was performed on samples collected at 0 and 72 hours to determine community taxonomy composition. OhyA orthologs in L. crispatus have distinct enzymatic activities, with only the OA-induced ortholog (OhyA9) exhibiting the ability to hydrate cis-9 double bonds of OA and related cis-9-U-LCFAs. The results of the competition assays showed that 1) 10-HSA effectively promotes L. crispatus dominance alone or with MTZ 2) 10-HSA selectively promotes the growth of only L. crispatus (an ohyA9-harboring species) and not other non-ohyA9-harboring species (e.g. (e.g. L. iners and Prevotella amnii) whereas 3) oleic acid may benefit non-susceptible FGT species regardless of their ohyA9-harboring status (FIG. 10). Selective 10-HSA growth promotion of L. crispatus supports the use of 10-HSA as an adjunctive therapy to promote L. crispatus dominance.

### Example 4: Diverse LCFA Metabolites Inhibit L. iners and BV-Associated Species

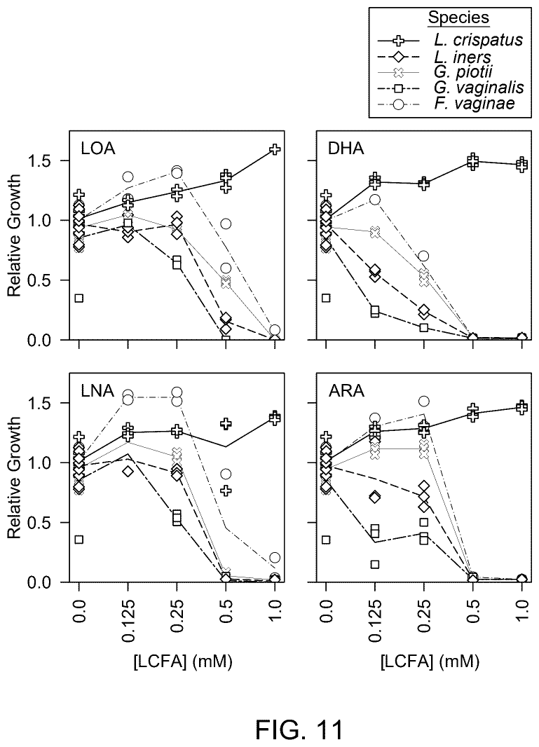

Growth of L. crispatus, L. iners, G. piotii, G. vaginalis, and F. vaginae was accessed with increasing amounts of linoleic acid (LOA), linolenic acid (LNA), cis-4,7,10,13,16,19-docosahexaenoic acid (DHA), and arachidonic acid (ARA). Results showed that U-LCFAs selectively inhibit L. iners along with certain BV-associated species and robustly promote growth of L. crispatus due to fatty acid response mechanisms that are uniquely conserved in L. crispatus and other non-iners FGT Lactobacillus species (FIG. 11).

### Example 5: LCFA Combinations Improve Efficacy of L. crispatus Growth, L. iners Growth Inhibition and Growth Inhibition of Diverse Vaginal Species Contributing to Bacterial Vaginosis

The materials and methods were as described herein above, with additional modifications to the culture conditions.

Bacterial liquid and solid culture media: To prepare MRSQC, BD Difco-formulated De Man, Rogosa and Sharpe (MRS) broth powder was prepared as per the manufacturer's instructions and then supplemented with L-cysteine (4 mM) and L. glutamine (1.1 mM). To prepare modified MRS with lipids removed, four parts MRS+CQ broth was combined with one part Cleanascite™ Lipid Removal Reagent (Biotech Support Group #X2555-10) and shaken (220 rpm) at room temperature for 10 min, then centrifuged at 2,000×g for 15 min. The media supernatant was collected and re-sterilized using a 0.22 uM PES filter.

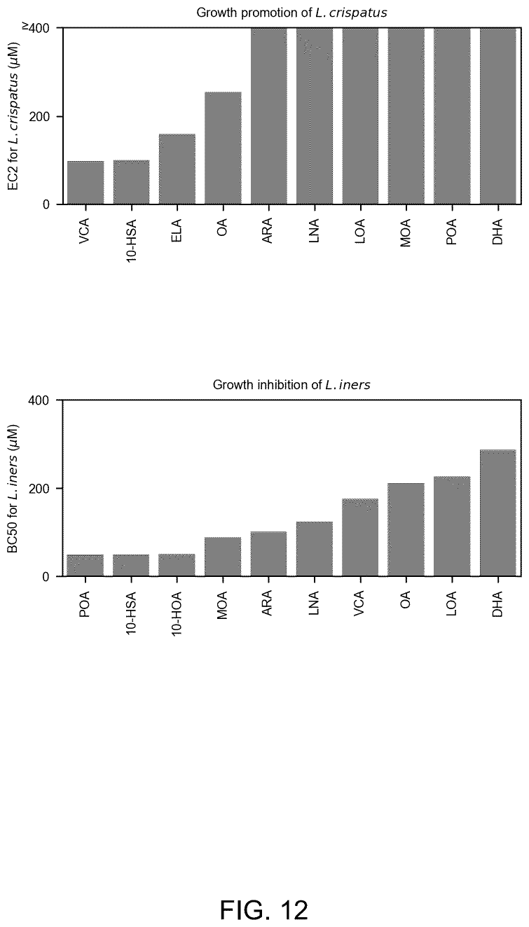

As shown in FIG. 12, cis-vaccenic acid, 10-hydroxystearic acid, elaidic acid, and oleic acid all demonstrate optimal L. crispatus growth promotion activity. Palmitoleic acid, 10-hydroxystearic acid, 10-hydroxy-12-octadecenoic acid, myristoleic acid, arachidonic acid, linolenic acid, oleic acid, and linoleic acid all demonstrate strong L. iners growth inhibitory activity. Any combination of these optimal L. crispatus growth promoters with strong L. iners growth inhibitors would serve as strong potential therapeutic compositions with improved efficacy over using a single LCFA alone.

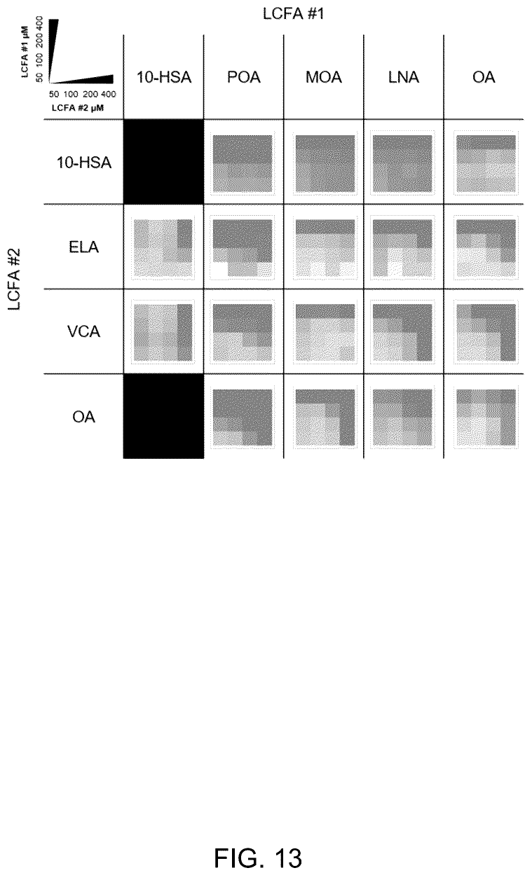

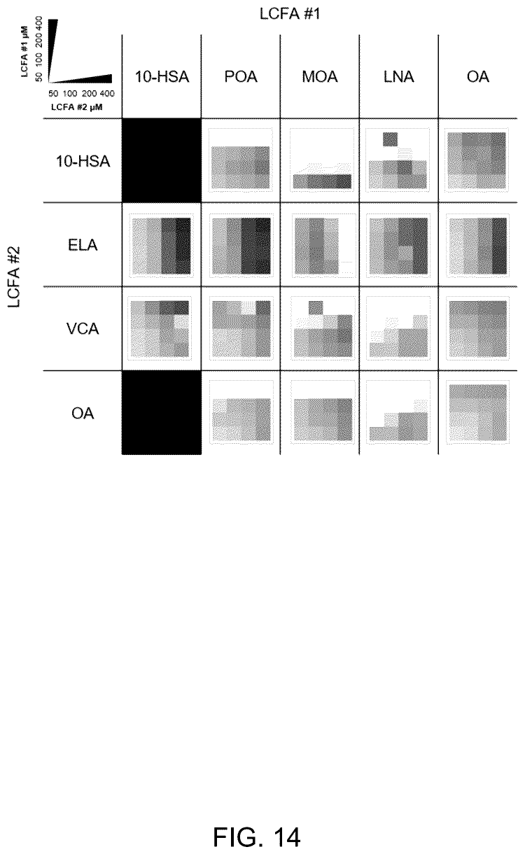

Most combinations show at least additive effects while surprisingly, synergistic L. iners growth inhibition for the following combinations was observed: elaidic acid with 10-hydroxystearic acid, elaidic acid with palmitoleic acid, elaidic acid with linolenic acid, elaidic acid with oleic acid, cis-vaccenic acid with palmitoleic, and cis-vaccenic acid with oleic acid (FIG. 13) and synergistic L. crispatus growth promotion for the following combinations was also observed: cis-vaccenic acid with 10-hydroxystearic acid and elaidic acid with linolenic acid (FIG. 14).

Additionally, using equimolar combinations of 10-hydroxystearic acid with myristoleic acid; elaidic acid with palmitoleic acid, elaidic acid with linolenic acid, cis-vaccenic acid with myristoleic acid, elaidic acid with 10-hydroxystearic acid; and, cis-vaccenic acid with 10-hydroxy-12-octadecenoic acid, all demonstrate optimal L. crispatus growth promotion activity. Notably 10-hydroxystearic acid with myristoleic acid, elaidic acid with palmitoleic acid, and elaidic acid with linolenic acid are more potent in a combination than when each LCFA is used alone.

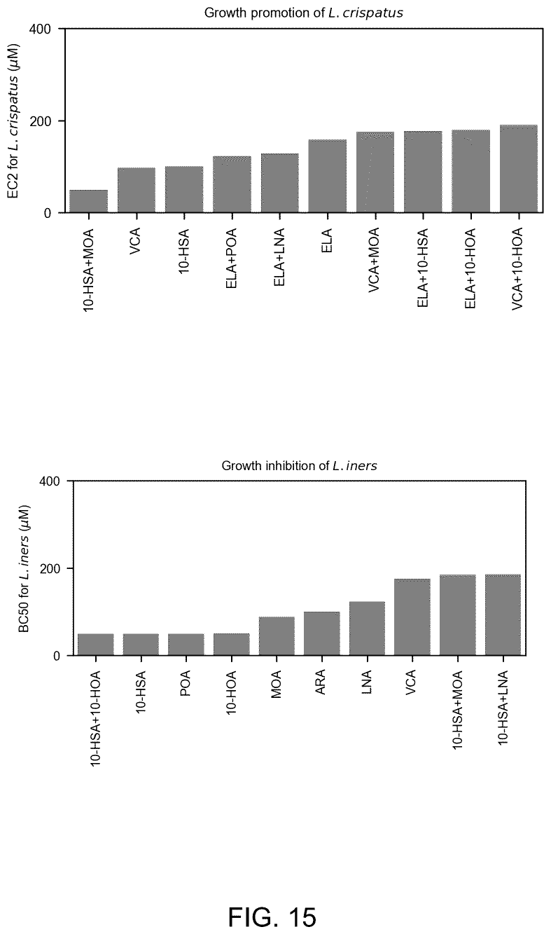

Equimolar combinations of 10-hydroxystearic acid with 10-hydroxy-12-octadecenoic acid, 10-hydroxystearic acid with myristoleic acid, and 10-hydroxystearic acid with linoleic acid all demonstrate strong L. iners growth inhibitory activity. Notably the equimolar concentration of 10-hydroxystearic acid with 10-hydroxy-12-octadecenoic acid performs equally as well as 10-hydroxystearic acid alone when the total LCFA molarity is held constant, indicating a synergistic effect (FIG. 15).

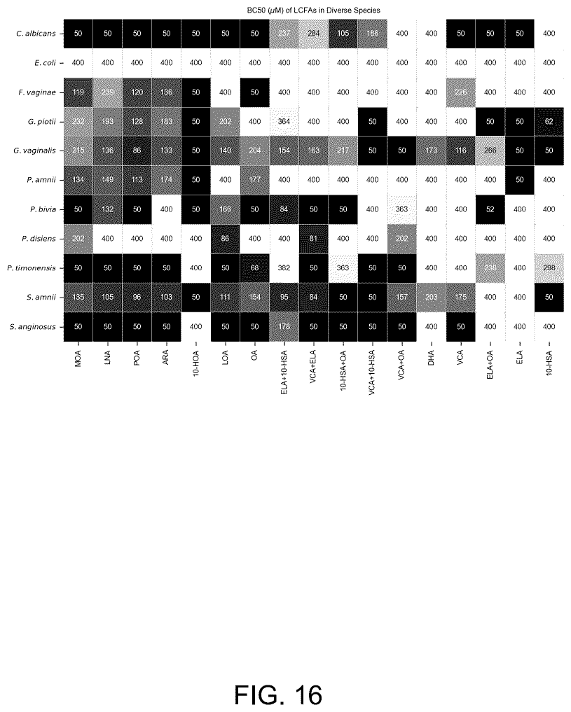

Growth inhibition of diverse vaginal species contributing to bacterial vaginosis upon LCFA treatment was also observed (FIG. 16). Notably synergistic effects for inhibition of these diverse species were observed for combinations, including elaidic acid with 10-hydroxystearic acid, cis-vaccenic acid with elaidic acid, and cis-vaccenic acid with 10-hydroxystearic acid.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating vaginal dysbiosis in a subject, said method comprising administering to the subject a composition comprising an active ingredient comprising or consisting of one or more unsaturated long chain fatty acids or metabolic derivative thereof in an amount and duration effective to increase vaginal colonization of non-Lactobacillus-iners species, thereby treating the vaginal dysbiosis in the subject.

2. A method of reducing the risk of a subject developing vaginal dysbiosis, said method comprising administering to the subject a composition comprising an active ingredient comprising or consisting of one or more unsaturated long chain fatty acids or metabolic derivative thereof in an amount and duration effective to increase vaginal colonization of non-Lactobacillus-iners species, thereby reducing the risk of the subject developing vaginal dysbiosis.

3. The method of claim 1 or 2, wherein the vaginal dysbiosis is bacterial vaginosis.

4. The method of any one of claims 1-3, wherein the one or more unsaturated long chain fatty acid or a metabolic derivative thereof is selected from oleic acid, myristoleic acid, palmitoleic acid, linoleic acid, linolenic acid, gamma-linolenic acid, cis-11-eicosenoic acid, erucic acid, elaidic acid, eicosapentaenoic acid, 11,14,17-eicosatrienoic acid, mead acid, nervonic acid, pinolenic acid, stearidonic acid, sapienic acid, cis-vaccenic acid, cis-4,7,10,13,16,19-docosahexaenoic acid, arachidonic acid, and their hydroxylated metabolic products 10-hydroxymyristic acid, 10-hydroxypalmitic acid, 10-hydroxystearic acid, 10-hydroxy-12-octadecenoic acid, and 13-hydroxy-9-octadecenoic acid or combinations thereof.

5. The method of claim 4, wherein the one or more unsaturated long chain fatty acid is oleic acid.

6. The method of claim 4, wherein the one or more metabolic derivative thereof is selected from 10-hydroxymyristic acid, 10-hydroxypalmitic acid, 10-hydroxystearic acid, 10-hydroxy-12-octadecenoic acid, and 13-hydroxy-9-octadecenoic acid or combinations thereof.

7. The method of claim 4, wherein the one or more unsaturated long chain fatty acid or metabolic derivative thereof comprises a combination of oleic acid and 10-hydroxystearic acid.

8. The method of any one of claims 1-7, wherein the composition comprising an active ingredient comprising or consisting of the one or more unsaturated long chain fatty acids or metabolic derivative thereof is administered following, or prior to, administration of an antimicrobial agent to the subject.

9. The method of claim 8, wherein the subject was previously administered the antimicrobial agent and is concurrently administered one or more additional doses of the antimicrobial agent with the unsaturated long chain fatty acid.

10. The method of claim 8 or 9, wherein the antimicrobial agent is metronidazole or clindamycin.

11. The method of claim 8, wherein the subject was previously administered the antimicrobial agent and is concurrently administered a different antimicrobial agent with the unsaturated long chain fatty acid or metabolic derivative thereof.

12. The method of any one of claims 1-11, wherein the method further comprises administration of one or more live biotherapeutics (LBPs).

13. The method of any one of claims 1-12, wherein the active ingredient consists of the one or more unsaturated long chain fatty acids or metabolic derivative thereof.

14. The method of any one of claims 1-13, wherein the composition is administered to the subject orally, rectally, or vaginally.

15. A method of treating a subject having Lactobacillus iners-dominant vaginal colonization, said method comprising administering to the subject a composition comprising an active ingredient comprising or consisting of one or more unsaturated long chain fatty acids or metabolic derivative thereof in an amount and duration effective to increase vaginal colonization of Lactobacillus crispatus and decrease vaginal colonization of Lactobacillus iners, thereby treating the subject.

16. A method of reducing the risk of a subject developing Lactobacillus iners-dominant vaginal colonization, said method comprising administering to the subject a composition comprising an active ingredient comprising or consisting of one or more unsaturated long chain fatty acids or metabolic derivative thereof in an amount and duration effective to increase vaginal colonization of Lactobacillu crispatus and decrease vaginal colonization of Lactobacillus iners, thereby reducing the risk of the subject developing Lactobacillus iners-dominant vaginal colonization.

17. The method of claim 15 or 16, wherein the one or more unsaturated long chain fatty acid or metabolic derivative thereof is selected from oleic acid, myristoleic acid. palmitoleic acid, linoleic acid, linolenic acid, gamma-linolenic acid, cis-11-eicosenoic acid, erucic acid, elaidic acid, eicosapentaenoic acid, 11,14,17-eicosatrienoic acid, mead acid, nervonic acid, pinolenic acid, stearidonic acid, sapienic acid, cis-vaccenic acid, cis-4,7,10,13,16,19-docosahexaenoic acid, arachidonic acid, and their hydroxylated metabolic products 10-hydroxymyristic acid, 10-hydroxypalmitic acid, 10-hydroxystearic acid, 10-hydroxy-12-octadecenoic acid, and 13-hydroxy-9-octadecenoic acid or combinations thereof.

18. The method of claim 17, wherein the one or more unsaturated long chain fatty acid is oleic acid.

19. The method of claim 17, wherein the one or more or metabolic derivative thereof is selected from 10-hydroxymyristic acid, 10-hydroxypalmitic acid, 10-hydroxystearic acid, 10-hydroxy-12-octadecenoic acid, and 13-hydroxy-9-octadecenoic acid or combinations thereof.

20. The method of claim 17, wherein the one or more unsaturated long chain fatty acid or metabolic derivative thereof comprises a combination of oleic acid and 10-hydroxystearic acid.

21. The method of any one of claims 15-20, wherein the composition comprising an active ingredient comprising or consisting of the one or more unsaturated long chain fatty acids or metabolic derivative thereof is administered following, or prior to, administration of an antimicrobial agent to the subject.

22. The method of claim 21, wherein the subject was previously administered the antimicrobial agent and is concurrently administered one or more additional doses of the antimicrobial agent with the unsaturated long chain fatty acid or metabolic derivative thereof.

23. The method of claim 21 or 22, wherein the antimicrobial agent is metronidazole or clindamycin.

24. The method of claim 21, wherein the subject was previously administered the antimicrobial agent and is concurrently administered a different antimicrobial agent with the unsaturated long chain fatty acid or metabolic derivative thereof.

25. The method of any one of claims 15-24, wherein the method further comprises administration of one or more live biotherapeutics (LBPs).

26. The method of any one of claims 15-25, wherein the active ingredient consists of the one or more unsaturated long chain fatty acids or metabolic derivative thereof.

27. The method of any one of claims 15-26, wherein the composition is administered to the subject orally, rectally, or vaginally.

28. A composition comprising an active ingredient consisting of one or more unsaturated long chain fatty acids or metabolic derivative thereof.

29. The composition of claim 28 wherein the one or more unsaturated long chain fatty acid or metabolic derivative thereof is selected from oleic acid, myristoleic acid, palmitoleic acid, linoleic acid, linolenic acid, gamma-linolenic acid, cis-11-eicosenoic acid, erucic acid, elaidic acid, eicosapentaenoic acid, 11,14,17-eicosatrienoic acid, mead acid, nervonic acid, pinolenic acid, stearidonic acid, sapienic acid, cis-vaccenic acid, cis-4,7,10,13,16,19-docosahexaenoic acid, arachidonic acid, and their hydroxylated metabolic products 10-hydroxymyristic acid, 10-hydroxypalmitic acid, 10-hydroxystearic acid, 10-hydroxy-12-octadecenoic acid, and 13-hydroxy-9-octadecenoic acid or combinations thereof.

30. The composition of claim 29, wherein the one or more unsaturated long chain fatty acid is oleic acid.

31. The composition of claim 29, wherein the one or more or metabolic derivative thereof is selected from 10-hydroxymyristic acid, 10-hydroxypalmitic acid, 10-hydroxystearic acid, 10-hydroxy-12-octadecenoic acid, and 13-hydroxy-9-octadecenoic acid or combinations thereof.

32. The composition of claim 29, wherein the one or more unsaturated long chain fatty acid or metabolic derivative thereof comprises a combination of oleic acid and 10-hydroxystearic acid.

33. The composition of any one of claims 28-32, further comprising one or more pharmaceutically acceptable excipients.

34. The composition of any one of claims 28-33, wherein the composition is formulated for oral, rectal, or vaginal administration.

35. The composition of any one of claims 28-34, wherein the active ingredient consisting of one or more unsaturated long chain fatty acids or metabolic derivative thereof is selected from a combination comprising or consisting of elaidic acid and 10-hydroxystearic acid, elaidic acid and palmitoleic acid, elaidic acid and linolenic acid, elaidic acid and oleic acid, cis-vaccenic acid and palmitoleic acid, cis-vaccenic acid and oleic acid, 10-hydroxystearic acid and 10-hydroxy-12-octadecenoic acid, cis-vaccenic acid and elaidic acid, cis-vaccenic acid and 10-hydroxystearic acid, cis-vaccenic acid and 10-hydroxystearic acid, elaidic acid and linolenic acid, 10-hydroxystearic acid and myristoleic acid, elaidic acid and palmitoleic acid, and elaidic acid and linolenic acid.

36. The method of any one of claims 15-27, wherein the active ingredient consisting of one or more unsaturated long chain fatty acids or metabolic derivative thereof is selected from a combination comprising or consisting of elaidic acid and 10-hydroxystearic acid, elaidic acid and palmitoleic acid, elaidic acid and linolenic acid, elaidic acid and oleic acid, cis-vaccenic acid and palmitoleic acid, cis-vaccenic acid and oleic acid, 10-hydroxystearic acid and 10-hydroxy-12-octadecenoic acid, cis-vaccenic acid and elaidic acid, cis-vaccenic acid and 10-hydroxystearic acid, cis-vaccenic acid and 10-hydroxystearic acid, elaidic acid and linolenic acid, 10-hydroxystearic acid and myristoleic acid, elaidic acid and palmitoleic acid, and elaidic acid and linolenic acid.

37. The method of any one of claims 1-13, wherein the active ingredient consisting of one or more unsaturated long chain fatty acids or metabolic derivative thereof is selected from a combination comprising or consisting of elaidic acid and 10-hydroxystearic acid, elaidic acid and palmitoleic acid, elaidic acid and linolenic acid, elaidic acid and oleic acid, cis-vaccenic acid and palmitoleic acid, cis-vaccenic acid and oleic acid, 10-hydroxystearic acid and 10-hydroxy-12-octadecenoic acid, cis-vaccenic acid and elaidic acid, cis-vaccenic acid and 10-hydroxystearic acid, cis-vaccenic acid and 10-hydroxystearic acid, elaidic acid and linolenic acid, 10-hydroxystearic acid and myristoleic acid, elaidic acid and palmitoleic acid, and elaidic acid and linolenic acid.
